(19) United States
(12) Patent Application Publication
(10) Pub. No.: US 2023/0183646 A1
(43) Pub. Date: Jun. 15, 2023

(54) METHODS AND PRODUCTION OF NOVEL PLATELETS

(71) Applicant: Stellular Bio, Inc., Watertown, MA (US)

(72) Inventors: Christian Peters, Watertown, MA (US); Lea Beauilieu, Watertown, MA (US); Brenden Smith, Watertown, MA (US)

(73) Assignee: Stellular Bio, Inc., Watertown, MA (US)

(21) Appl. No.: 17/985,641

(22) Filed: Nov. 11, 2022

Related U.S. Application Data
(63) Continuation of application No. PCT/US21/32646, filed on May 15, 2021.
(60) Provisional application No. 63/025,209, filed on May 15, 2020.

(51) Int. Cl. C12N 5/078

(52) U.S. Cl. CPC C12N 5/0644; C12N 2506/03

(57) ABSTRACT

The present disclosure provides methods for generating megakaryocytes and novel platelet variants from the same CD34+ progenitor stem cells, which comprises at least two stages: stage zero (0) comprising an expansion and maintenance stage of the CD34+ progenitor stem cells for a period ranging between 0 hours to 48 hours; and, stage one (I) comprising a differentiation phase wherein the differentiation phase comprises differentiating the CD34+ progenitor stem cells in step (i) for a period sufficient to generate substantially matured megakaryocytes. Novel platelet variants are produced by passaging the megakaryocytes, produced by the CD34+ progenitor stem cells, through a bioreactor or a fluidic device. Formulations comprising megakaryocytes and platelet variants derived from CD34+ progenitor stem cells and methods of their use are also disclosed.

### RELATED APPLICATIONS

This application is a continuation of PCT/US21/32646, filed May 15, 2021, which claims the benefit of and priority to U.S. Provisional Patent Application Ser. No. 63/025,209, filed May 15, 2020; the entire contents of all of which are incorporated by reference herein in their entireties.

### FIELD

The disclosure is generally directed to improved methods of making anucleated cells from progenitor cells as well as making novel anucleated cells from the same progenitor cells. Specifically, the present disclosure is directed to improved methods of making platelets from progenitor cells as well as to the making of platelet variants from the same progenitor cells.

### BACKGROUND

Appreciation of megakaryocyte (MK) biology is critical to understanding and treating diseases of defective platelet (PLT) production and function. However, the sourcing and differentiation of functional megakaryocytes (MKs) in culture remains rate-limiting for the production of commercially viable, safe, and functional platelets (PLTs) for clinical applications.

Many fundamental MK cellular processes, including differentiation, proliferation, pro-platelet (proPLT) formation, and PLT production, remain incompletely understood.

Large-scale generation ex vivo of PLTs suitable for clinical use remains a lofty goal not only in transfusion medicine but also for treating patients with low PLT counts due to chemotherapy, stem cell transplants, acute injury, infection, and genetic disorders. However, the short shelf life of PLTs leaves the national supply vulnerable to shortages due to decreased donations during cold weather, flu season, or summer vacation, as well as less predictable events such as public health emergencies, natural disasters, or radiation disasters. Donor-derived PLTs also present the risks of transmitting known or emerging pathogens or patients developing PLT refractoriness after multiple transfusions. Thus, a reliable method of large-scale, ex vivo PLT or the production of platelet variants would increase resilience of the PLT supply while mitigating the medical concerns arising from the use of human donors.

To address the insufficient supply of ex vivo manufactured platelets, disclosed herein are improved methods of platelet productions from progenitor cells (e.g., CD34+ progenitor cells) as well as preparation of novel platelet variants, which can be readily scaled up for their use in clinical applications.

### SUMMARY

Megakaryocytes (MKs) reside primarily in the bone marrow and to some extent in the lung, comprising less than 0.1% of the myeloid cell population, making them difficult to obtain and isolate in large numbers. As a result, efforts to study MK biology have shifted toward MKs differentiated from human pluripotent stem cells (hPSCs) in cell culture. While hPSC-derived MKs have proven to be valuable tools in studying MK biology, there remains a lack of consensus on a rapid method to rapidly generate a pure and consistent population of MKs from hPSCs that can produce functional platelets for clinical use.

Described herein are methods that advantageously enhance CD34+ progenitor cell (e.g., CD34+ peripheral blood cells) differentiation into substantially pure and consistent population of MKs that can be utilized to produce platelets or are processed through a bioreactor or a fluidic device to produce novel platelet variants, which are variants of bone morrow derived platelets. The platelets produced by the rapid methodologies of the present disclosure as well as the novel platelet variants are suitable for treating a disease or a disorder where platelets are needed to cure such disorder.

Another advantage is the scale up of the MKs in a bioreactor to produce platelet variants or derivatives thereof. Further advantages are provided by the drug delivery applications of CD34+ progenitor cell-derived platelets or the CD34+ progenitor cell bioreactor-derived platelet variants, demonstrating that they can efficiently uptake and release (i.e., cargo carrying capacity) drugs (e.g., therapeutic proteins, nucleotides, small molecules or antibody drugs such as ipilimumab, a monoclonal antibody targeting CTLA-4). They also can advantageously be conjugated to greater number of drugs molecules for the drug delivery. They also can advantageously be genetically engineered to express a molecule of interest (e.g., protein, DNA, siRNA) for their therapeutic use. The biodistribution as well as many of the functional properties of the bioreactor-derived platelet variants in vivo mimic that of donor PLTs.

Some embodiments of the present disclosure include methods of differentiating human CD34+ progenitor cells to generate MKs. The CD34+ progenitor cell can be derived from, but not limited to, CD34+ hematopoietic stem cells from umbilical cord blood or peripheral blood (HSCs), or adipose-derived mesenchymal stem cells. The advantages of using CD34+ progenitor cells include manipulating these cells to unlimited expansion capacity, ability to source a cGMP-compliant stem cell line, scalability of process, and reproducibility. Additionally, they provide the opportunity to control the cost of generating MKs.

In some embodiments, the CD34+ megakaryocytic progenitors (e.g., umbilical cord blood or peripheral blood cells cells), megakaryocytes, proplatelets, preplatelets derived therefrom, which produce platelet variants when passed through a bioreactor or a fluidic device, can be modified to express a protein of interest (including polypeptides or peptides of interest). Such modifications can take place at the CD34+ progenitor stem cell level (e.g., umbilical cord blood or peripheral blood cells), in megakaryocytes derived from CD34+ progenitor stem cell, in proplatelets, preplatelets or in the platelet variants or at any other level during the generation of the platelet variants. Genetic engineering of megakaryocytes or megakaryocytic progenitors differentiated from a genetically engineered human CD34+ progenitor cells or cell lines, where the genetic manipulation leads megakaryocytes or megakaryocytic progenitor cells to express a protein or a polypeptide of interest are also encompassed by the disclosure.

In some embodiments, the platelets or the platelet variants or derivatives thereof on their own, or genetically engineered, or bioconjugated or in their cargo carrying capacity can deliver a protein of interest systemically or at first diseased location, generally the site of a disease where the platelet variants are administered or to a second diseased location, different form the site where the platelet variants or their derivatives are administered. In some embodiments, they can be administered locally, i.e., adhere and aggregate at an injury site or a diseased site at the first location (i.e., are administered locally to mitigate or eliminate the injury (e.g., bleeding) or the disease (e.g., neoplasm, autoimmune or anti-inflammatory diseases, among others)). Other advantages are that they can travel through blood flow without inducing immunogenicity, are not cancerous cells; and do not exhibit uncontrolled growth or tumor formation in vivo. They also provide the advantage of carrying and delivering to target cells higher drug payloads (e.g., genetically engineered payloads, or conjugated payloads or infused payloads) because of their large surface area as compared to other payload carrying agents such as an antibody in an antibody drug conjugate (ADC).

Platelets variants or derivatives thereof that can be derived from genetically engineered CD34+ progenitor cells or megakaryocytes derived therefrom can be produced in a device or a system that supports a biologically active environment e.g., bioreactors or fluidic devices. For example, platelet variants can be generated in a bioreactor that mimics the in vivo environment for endogenous platelet generation in humans.

Exemplary vectors that can be used to genetically engineer CD34+ progenitor cells or megakaryocytes that produce the platelet variants or derivatives of the present disclosure, such that the platelet variants produce a genetic product intended to be produced in the platelet variants (e.g., CTLA4 antibody or a fragment thereof) include, but are not limited to, plasmids, retroviral vectors, lentiviral vectors, adenovirus vectors, adeno-associated virus (AAV) vectors, a herpes simplex virus vectors, poxvirus vectors, or baculoviral vectors. The vector comprising the nucleic acid molecule of interest may be delivered to the cell (e.g., peripheral blood cell, megakaryocytic progenitor, or megakaryocyte) via any method known in the art, including but not limited to transduction, transfection, infection, and electroporation.

In some embodiments, the platelets or the platelet variants or derivatives thereof may be co-administered with ex-vivo derived endogenous cells that are autogenic (e.g., bone marrow derived donor platelets). Here, the ex-vivo derived autogenic endogenous cells may be derived from a healthy donor. In some embodiments, prior to co-administering, the ex-vivo derived autogenic endogenous cells may be modified in a manner that complements the platelets or the platelet variants in alleviating the harmful role of a platelet-related diseases or disorders or where the platelets or the platelet variants or derivatives thereof can be administered or delivered. For example, when the platelets or the platelet variants of the present disclosure are co-administered with ex-vivo derived autogenic endogenous cells from other sources, the platelets or the platelet variants or derivatives thereof cumulatively assist in restoring the physiological role of the autogenic endogenous cell population affected by the disease or disorder.

In some embodiments, the platelets or the platelet variants or derivatives thereof of the present disclosure may be co-administered with non-natural extracellular vesicles (EVs) made in vivo or ex vivo or in vitro by any eucaryotic cell. Extracellular vesicles (EVs) comprise microvesicles (MVs) or exosomes or a combination thereof, are smaller in size as compared to the platelets or the platelet variants or derivatives thereof, and are biologically active. The extracellular vesicles (EVs) function as a transport and delivery system for bioactive molecules, play a role in hemostasis and thrombosis, inflammation, malignancy infection transfer, angiogenesis, and immunity. Thus, in some embodiments, EVs may complement the platelets or the platelet variants or derivatives thereof and their combinational use is an even richer resource for the platelets or the platelet variants or derivatives thereof-based therapeutic applications.

In some embodiments, the EVs comprise exosomes, approximately ranging between 65 nm to about 10 μm in diameter carrying multifarious molecules such as proteins, lipids, and RNAs either on their surface or within their lumen. Exosomes play a role in stimulating tissue regeneration, in many in vitro and in vivo models, demonstrating that they can confer proangiogenic, proliferative, antiapoptotic and anti-inflammatory actions through transporting RNA and protein cargos. Thus, in some embodiments, exosomes make it even a richer resource for the platelets or the platelet variants or derivatives thereof-based therapeutic applications.

In some embodiments, the EVs comprise microvesicles (MVs), approximately ranging between 65 nm to about 10 μm in diameter, carrying multifarious molecules such as proteins, lipids, and RNAs either on their surface or within their lumen. MVs play a role in stimulating tissue regeneration, in many in vitro and in vivo models, demonstrating that they can confer proangiogenic, proliferative, antiapoptotic and anti-inflammatory actions through transporting RNA and protein cargos. Thus, in some embodiments, MVs make it even a richer resource for the platelets or the platelet variants or derivatives thereof-based therapeutic applications.

In some embodiments, the present disclosure provides a pharmaceutical composition comprising the platelets or the platelet variants or derivatives thereof of the present disclosure and pharmaceutically acceptable carriers, various diluents, fillers, salts, buffers, stabilizers, solubilizers, and other materials well known in the art. In some embodiments, the pharmaceutical composition comprises a second or a third therapeutic agent.

In some embodiments, the present disclosure provides a method of treating a patient suffering from a disease or disorder (e.g., e.g., immunoinflammatory disorder, a metabolic disorder, a neoplastic disorder, an autoimmune disorder, viral or bacterial-induced disorder or defects in primary hemostasis), the method comprising administering to the patients the platelets or the platelet variants or derivatives thereof of the present disclosure, alone or with other therapeutic drugs thereby causing amelioration of or treatment of the disease or disorder.

In some embodiments, the present disclosure provides a method of treating a patient suffering from a disorder in which the disorder is inclusive of a ligand or an antigen that has affinity for a receptor on the platelets or the platelet variants or derivatives thereof of the present disclosure. In some embodiments, the platelets or the platelet variants or derivatives thereof can bind to the ligand or antigen of interest thereby ameliorating or treating a disorder by blocking the activity of the ligand or antigen, the method comprising administering to the patients the platelets or the platelet variants or derivatives thereof of the present disclosure expressing a receptor that will specifically bind to the ligand or antigen with relatively high affinity, the receptor-ligand/antigen interaction causing the removal a molecule bearing the diseased ligand or antigen to thereby ameliorate or eliminate the disorder caused by the disease-bearing molecule. In some embodiments, receptors on the platelets or the platelet variants or derivatives thereof are conjugated to a cytotoxic agent, which are then delivered to a diseased cell bearing the ligand/antigen of interest. In some embodiments, cytotoxic agents are carried as a deliverable cargo by the platelets or the platelet variants or derivatives thereof, which are then delivered to a diseased cell bearing the ligand/antigen of interest. In some embodiments, the genetically engineered platelets or the platelet variants deliver a cytotoxic agent to a diseased molecule bearing the ligand/antigen of interest. In some embodiments, the therapeutic effect is the removal of the disease molecule to ameliorate or treat a disease or a disorder caused by the molecule bearing the ligand/antigen of interest.

In some embodiments, the present disclosure provides a method of therapeutically treating a mammal having a tumor comprising targeting donor platelets in a cancer microenvironment with platelets or the platelet variants or derivatives thereof that act as decoys (e.g., platelets or the platelet variants or derivatives thereof carrying drug payloads (e.g., antibodies) are contacted by a tumor metastasizing cells thinking it to be endogenous donor platelet and the drug on the platelets or the platelet variants or derivatives thereof kills the metastasizing cells), the method comprising administering to the mammal a therapeutically effective amount of platelets or the platelet variants or derivatives thereof that act as decoys or are programmed to act as decoys, the decoys deceiving the metastasizing tumor cells from believing that they are the donor platelets, thereby effectively ameliorating or treating the tumor by killing the tumor cells or preventing the tumor cells from metastasizing. In some embodiments, the platelets or the platelet variants or derivatives thereof carry drug payloads. For example, platelets or the platelet variants or derivatives thereof are conjugated to a cytotoxic agent or the platelets or the platelet variants or derivatives thereof are conjugated to a growth inhibitory agent or the platelets or the platelet variants or derivatives thereof are genetically engineered platelets or the platelet variants which produce a cytotoxic agent or a growth inhibitory agent.

In some embodiments, the platelets or the platelet variants or derivatives thereof carry growth factors or cytokines for tissue regeneration. In some embodiments, the platelets or the platelet variants or derivatives thereof, deliver proteins expressed in their granules (e.g., alpha-granules), or deliver proteins expressed on their cell surface or deliver proteins expressed in their transmembrane domains, or deliver proteins packaged in platelet variant-produced microsomes or exosomes.

In some embodiments, the present disclosure provides diagnostic reagents where the receptors or ligands or antigens on the cell surface of the platelets or the platelet variants or derivatives thereof are labeled. The label is selected from the group consisting of a radiolabel, a fluorophore, a chromophore, an imaging agent, and a metal ion. Labelling techniques are well known to one of skill in the art.

In some embodiments, the present disclosure provides a kit comprising the platelets or the platelet variants or derivatives thereof of the present disclosure described herein. Here, platelets or the platelet variants or derivatives thereof are engineered to recognize one or more viral receptors or protein for an early diagnostic of bacterial or viral infections such as coronavirus, or Ebola virus, or any other virus if the platelets or the platelet variants or derivatives thereof recognize such viral receptors or proteins (e.g., viral adhesion of entry proteins). In some embodiments, they can be used to detect any toxic molecule for their removal or treatment as disclosed in co-pending U.S. application Ser. No. 17/213,796, filed on Mar. 26, 2021, which is expressly incorporated herein by reference it its entirety.

### BRIEF DESCRIPTION OF DRAWINGS

The present disclosure will be described in the detailed description which follows, in reference to the noted plurality of drawings by way of non-limiting examples of exemplary embodiments, in which like reference numerals represent similar parts throughout the several views of the drawings, and wherein:

FIGS. 1A-1F show a schematic representation of MK processing and characterization of mature MKs. FIG. 1A is a schematic showing each step in the processing of CD34+ donor cells beginning with isolation from human donors to drug loading of platelet product. FIG. B shows MKs on days 7, 8 and 9 as they are processed over time. IG. 1C shows flow cytometry analysis of exemplary CD34+ progenitor cell-derived MK cultures displaying the percent-positive populations of CD34+ and cell viability (measured as percent PI-negative cells) from differentiation days 2-12. The grey box indicates the optimal harvest window (days 7-9) where cells are low in CD34+ signal and high in viability and MK markers. FIG. 1D shows a representative histogram displaying the ploidy distribution of CD61+ cells on day 9 and a bar graph showing average ploidy size on days 8 and 9. Cell ploidy analyzed using PI. (n=6 biological replicates). FIG. 1E is a bar graph showing representative percent-positive populations of the mature megakaryocyte markers CD61, CD42a, and CD42b on day 9 by flow cytometry analysis. Viability is measured as percent PI-negative cells. FIG. 1F shows transmission electron microscope micrographs of MKs at increasing magnifications (890×, and 9300×, respectively). Arrows indicate standard MK characteristics (multilobed nuclei, microtubule rings, alpha-granules, invaginated membrane system, and Golgi stacks). Scale bars are 6 μm (890×), and 600 nm (9300×), respectively.

FIGS. 2A-2C show exemplary CD34+ progenitor cell-derived MKs produce proPLTs. FIG. 2A shows brightfield images demonstrating the presence of proPLT-producing MKs from culture day 7 to day 9. FIG. 2B shows confocal micrographs of proPLT-producing MKs on day 8. F-actin was labeled using phalloidin-488 and nuclei were labeled using DRAQ5. FIG. 2C shows confocal micrographs of proPLT-producing MKs on day 8. Cells were visualized using specific antisera for β1-tubulin and counterstained with the nuclear dye DRAQ5. Arrows indicate proplatelets/platelets and the scale bar represents 25 μm.

FIGS. 3A-3C show granule localization in proPLT-producing MKs and the presence of cytokines in lysate from CD34+ cells. FIG. 3A shows confocal micrographs demonstrating the punctate presence of alpha-granule markers platelet factor 4 (PF4) and von Willebrand factor (VWF) in proplatelet-producing cultures. DRAQ5 was used for nuclear visualization. CD61 staining indicates MK maturity. FIG. 3B shows visualization of the dense granule markers serotonin and LAMP1, in addition to overlays with DRAQ5 and CD61. FIG. 3C shows prepared samples of CD34+ progenitor cell-derived MK lysate and commercially sold human PLT lysate (PLTMax, Sigma) were analyzed using Luminex technology by Eve Technologies Corporation. Cytokine concentration in each sample was calculated from its fluorescent intensity, based on a standard curve. A cytokine was designated as “present” if the concentration was above zero, and “not present” if these conditions were not satisfied.

FIGS. 4A-4E show exemplary CD34+ progenitor cell-derived MKs produce functional PLTs when seeded in a novel bioreactor. FIG. 4A is a schematic of platelet production in the blood vessel. FIG. 4B is a cross sectional schematic of the two-channel bioreactor. A porous membrane separates the two channels. MKs are seeded in the top channel, and PLTs and proPLTs generated by the MKs pass through the membrane pores into the second channel, from which they can be retrieved for analysis. FIG. 4C is an image showing a bioreactor and pump system for continuous recirculation. FIG. 4D illustrates a graph showing that the sheer rate in the PLT channel stays constant throughout the channel length and a graph showing that the change in pressure between the PLT and MK channels stays constant across their length. FIG. 4E shows the 16-channel version of the bioreactor and fluorescent images showing even seeding of MKs throughout its channels and their lengths.

FIGS. 5A-5E show exemplary CD34+ progenitor cell-derived MKs produce functional PLTs when seeded in a novel bioreactor. FIG. 5A shows a graph of a number of PLTs generated in the novel bioreactor over a continuous period of three hours. FIG. 5B is a bar chart indicating total PLTs produced in a bioreactor (BioR PLTs) relative to PLTs present in MK input volume (Static PLTs), (n=3). FIG. 5C shows activation of PLTs by 20 μm TRAP6 measured by flow cytometry. Two plots show resting and activated platelets by CD62p signal (13.0% and 60.2% respectively). FIG. 5D shows a micrograph of bioreactor-derived platelets labeled with F-actin. FIG. 5E shows PLTs spread on coated glass coverslips stained for F-actin and P-selectin.

FIGS. 6A-6F show biodistribution of CD34+ PLTs resembles isolated human donor PLTs. FIG. 6A shows NCG mice that underwent engraftment with dye-labeled human donor PLTs. FIG. 6B shows NCG mice that underwent engraftment with dye-labeled CD34+ derived bioreactor platelets. Mice (n=5 per condition) received a single 200 μL dose of PLTs administered via tail vein and were imaged at 15-minute intervals for 1 hour. This was confirmed ex vivo as both the donor PLTs and the CD34+ PLTs primarily homed to the liver, though detectable engraftment was also observed in the lung, spleen, kidney, and heart as shown in FIGS. 6C and 6D. Additionally, fluorescent readings from tissues in FIGS. 6C and 6D were normalized to readings from ex vivo tissues of dye-free applications of PLTs for the given conditions and then compared to one another, as shown in the bar graph in FIG. 6E. The percent of biodistribution and tissue weight was compared and resulted in significant differences in the spleen, as shown in the bar graph in FIG. 6F. All imaging was conducted on an IVIS imager and examined by PerkinElmer Living Image software.

FIGS. 7A-7E show the characterizing drug-loading capabilities of CD34+-derived platelets or platelet variants. Human CTLA4 monoclonal antibody, ipilimumab, was labeled with NHS-ester Cy5.5 and loaded into CD34+ bioreactor-derived platelets or novel variants thereof at varying drug and PLT/PLT-variant concentrations. FIG. 7A shows graphs illustrating the ability of the platelets or variants thereof to load and retain drug after washes was measured by flow cytometry examining MFI. FIG. 7A shows cells loaded with drug at concentrations of Opg/PLT or variant, 100 pg/PLT or variant, and 300 pg/PLT or variant during an initial loading, single spin, and final spin. FIG. 7B is a bar graph showing retention of the drug across these washes quantified via plate reader against a standard curve of known drug. When 5×10⁵ CD34+ PLTs or platelet variants were exposed to increasing concentrations of Cy5.5-labeled Ipilimumab, the increased retention of drug directly related to increased drug concentration was observed, as shown in the graph in FIG. 7C. Upon titration varying both the concentration of PLTs or platelet variants and drug, it was noted that each concentration of drug approached a saturating dose via plots of MFI vs the input ratio of [pg of Ipilimumab] per PLT or platelet variant (pg/PLT or platelet variant). FIG. 7D illustrates CD34+ PLTs or platelet variant exposed to either 10, 100, 300, or 600 pg/PLT at concentrations of PLTs. FIG. 7E shows confocal micrographs of PLT or platelet variant-loaded Ipilimumab in relation to platelet factor 4 (PF4) and the membrane protein CD61.

FIG. 8 shows characterization of mature megakaryocytes analyzed by flow cytometry. They were first gated to be propidium iodide negative, indicating live cells. The live cells were then gated to be CD61 positive. The CD61 positive cells were found to be either CD61/CD42a/CD42b triple positive, indicating mature megakaryocytes, or CD61/CD42a positive/CD42b negative indicating a pre-apoptotic population. Any CD61 positive cells outside of those populations were considered immature megakaryocytes.

FIG. 9 shows flow cytometry scatter plots describing the rationale of the analysis of donor platelets. Product is initial separated by DNA positive events as stained by DRAQ5. CD61 positive events are then selected from the DRAQ negative events to represent platelet like particles. Additional gates for platelet specificity include a platelet size gate, CD42a+, and Calcein-AM positive events.

FIG. 10 shows flow cytometry scatter plots describing the rationale of the analysis of bioreactor product. Gates were created based on donor platelets. Product is initially separated by DNA positive events as stained by DRAQ5. CD61 positive events are then selected from the DRAQ negative events to represent platelet variants. Additional gates for platelet variant specificity include a platelet size gate, CD42a+, and Calcein-AM positive events.

FIG. 11 shows static platelet flow cytometry analysis. Supernatant from static cultures were analyzed for spontaneous platelet production. Platelet sized events were selected and analyzed for the presence of the nuclear stain, DRAQ5. DNA negative events scrutinized for the presence of CD61 and CD42a expression. The resulting platelet population was further characterized for activation status by CD62p surface expression.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “variant” or “variants” as interchangeably used herein is inclusive of a structural makeup of the platelet variants that is comparable to the structural make-up of the platelets, either in resting or in their activated stages. For example, platelet variants derived from CD34+ progenitor cells and the platelets derived from the same CD34+ progenitor cells have the same structural makeup, for example, may have m % CD36, or n % CD42a, or o % CD42a-b-d, or p % CD61, or q % CD62p, or x % CD63 receptors, where the m %, n %, o %, p %, q % x % are the same (i.e., have equal values) between the variants and the platelets. In other words, structurally platelet variants derived from the CD34+ progenitor cells may substantially be same to platelets derived from the same CD34+ progenitor cells, yet manifest the advantages over endogenous platelets or platelets derived from other source. In some embodiments, platelet variants, although derived from the same source (i.e., CD34+ progenitor cells) may manifest structural variety, structural deviation, or structural differences from the platelets. As non-limited examples, variants may structurally have greater than 3.6% of CD62p compared to platelets or variants may have less than 100% of CD42a as compared to the platelets or may have less than 99% of calcein as compared to platelets. In other non-limiting examples, structurally, variants may comprise greater than an average of CD63 receptors as compared to the platelets. In some embodiments, a variant comprises less than an average of CD36 receptor as compared to the platelet or comprise less than an average of CD42b receptor as compared to the platelets or comprise less than an average of glycoprotein VI receptor as compared to the platelets even though they are derived from the same CD34+ progenitor cells. When platelets are made from megakaryocytes by passaging the megakaryocytes through a bioreactor, a fluidic device or the like, they are interchangeably referred to as PLC, PLCs, artificial platelets or platelet variants.

As used herein, the terms “treat,” treating,” “treatment,” and the like refer to reducing or ameliorating a disorder and/or symptom associated therewith. It will be appreciated that, although not precluded, treating a disorder or condition does not require that the disorder, condition or symptoms associated therewith be eliminated.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

By “biologic sample” is meant any tissue, cell, fluid, or other material derived from an organism.

By “capture reagent” is meant a reagent that specifically binds a nucleic acid molecule or polypeptide to select or isolate the nucleic acid molecule or polypeptide.

By “cellular composition” is meant any composition comprising one or more isolated cells.

By “cell survival” is meant cell viability.

By “effective amount” is meant the amount of an agent required to produce an intended effect.

By “fragment” is meant a portion of a polypeptide or nucleic acid molecule.

This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The term “Non-natural” as used herein refers to manufactured, created, or constructed by human beings, artificial, or mimicking something that exists in nature.

The term “structure” as used herein refers to receptor distributions on the artificially produced (non-natural) platelet variants or the platelet like cells and as they connect to each other. Simply stated the platelet variants may have structural makeup that are unique not found in nature, because they are made ex-vivo and/or in vitro. As used herein the term structure is also used in context of cell size, cell dimension, surface area or volume.

The terms “resting stage” or “resting” refer to a stage in which cells are circulating in blood vessels without forming interactions with non-activated vascular endothelium under normal physiologic conditions.

The terms “derivative” or “derivatives”, as used herein, refer to genetically engineered CD34+ progenitor cells and derivatives thereof, inclusive of platelet variant producing precursor cells (e.g., CD34+ progenitor cells genetically engineered in a manner such that that the platelet variants produced by these precursor cells produce a molecule of interest in the platelet variants). The term derivatives is also inclusive of bioconjugates made from platelet variants or derivatives therefrom, or any other modification of the platelet variants described herein.

These terms, as used herein, can also refer to genetically engineered platelets or platelet variants produced by the genetically engineered CD34+ progenitor cells or the genetically engineered platelets or platelet variants. Derivatives are also inclusive of bioconjugates of platelets or platelet variants or bioconjugates of genetically engineered platelets or platelet variants. Derivatives are also inclusive of cargo carrying platelets or platelet variants or cargo carrying genetically engineered platelets or platelet variants. For example, the platelets or platelet variants can be first subjected to genetic engineering, then their cargo carrying capacity is utilized. In other words, the term derivative is inclusive of any modification, genetic, chemical or a combination thereof or otherwise of the platelets or platelet variants, genetically engineered platelets or platelet variants.

As used herein, the term “progenitor cells” refers to CD34+-derived progenitor cells, such as preMKs, MKs, proplatelets, preplatelets. It is also inclusive of “pluripotent stem cells”, which includes embryonic stem cells, embryo-derived stem cells, and induced pluripotent stem cells and other stem cells having the capacity to form cells from all three germ layers of the body, regardless of the method by which the pluripotent stem cells are derived. Pluripotent stem cells are defined functionally as stem cells that can have one or more of the following characteristics: (a) be capable of inducing teratomas when transplanted in immunodeficient (SCID) mice; (b) capable of differentiating to cell types of all three germ layers (e.g., can differentiate to ectodermal, mesodermal, and endodermal cell types); or (c) express one or more markers of embryonic stem cells (e.g., express Oct4, alkaline phosphatase. SSEA-3 surface antigen, SSEA-4 surface antigen, SSEA-5 surface antigen, Nanog, TRA-1-60, TRA-1-81, SOX2, REX1. Progenitor cells also include “megakaryocytic progenitor” (preMK), which refers to a mononuclear hematopoietic cell that is committed to the megakaryocyte lineage and is a precursor to mature megakaryocytes. Megakaryocytic progenitors are normally found in (but not limited to) bone marrow and other hematopoietic locations, but can also be generated from pluripotent stem cells, such as by further differentiation of hemogenic endothelial cells that were themselves derived from pluripotent stem cells.

The term “agonist” as used herein refers to a substance which initiates a physiological response when combined with a receptor. “Agonist Activated”, as used herein, refers to cell receptor or ligand activation induced by a receptor specific agonist. Agonists activate cells by binding to their respective receptors or ligands on a cell.

The term “antagonist” refers to any agent or entity capable of inhibiting the expression or activity of a protein, polypeptide portion thereof, or polynucleotide. Thus, the antagonist may operate to prevent transcription, translation, post-transcriptional or post-translational processing or otherwise inhibit the activity of the protein, polypeptide, or polynucleotide in any way, via either direct or indirect action. The antagonist may for example be a nucleic acid, peptide, or any other suitable chemical compound or molecule or any combination of these. Additionally, it will be understood that in indirectly impairing the activity of a protein, polypeptide of polynucleotide, the antagonist may affect the activity of the cellular molecules which may in turn act as regulators or the protein, polypeptide or polynucleotide itself. Similarly, the antagonist may affect the activity of molecules which are themselves subject to the regulation or modulation by the protein, polypeptide of polynucleotide.

The term “donor platelets” refer to bone marrow derived platelets physiologically generated in a mammalian (e.g., human) body.

The term “average” as used herein is a number expressing the central or typical value in a set of data, in particular the mode, median, or (most commonly) the mean, which is calculated by dividing the sum of the values in the set by their number. It also refers to a single value (such as a mean, mode, or median) that summarizes or represents the general significance of a set of unequal values.

The term “drug”, “agent” or “compound” as used herein refers to a biological product or a chemical entity or combination of biological product and chemical entities, administered in a therapeutic amount to a person to treat or prevent or control a disease or condition. The biological product or the chemical entity is inclusive of but not limited to, antibodies or fragments thereof, a low molecular weight compound, but may also be a larger compound, for example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The term “agent” “therapeutic composition,” or “therapeutic agent” can be used interchangeably and refers to a therapeutic agent. Agent may be selected from one or more of proteins; peptides; aptamers; antibodies; or fragments thereof; chemicals; small molecules; nucleic acid sequences; nucleic acid analogues. A nucleic acid sequence may be RNA or DNA, and may be single or double stranded, and can be selected from; nucleic acid encoding a protein of interest, oligonucleotides, nucleic acid analogues, for example peptide-nucleic acid (PNA), pseudo-complementary PNA (pc-PNA), locked nucleic acid (LNA), etc. Such nucleic acid sequences include, for example, but not limited to, nucleic acid sequence encoding proteins, for example that act as transcriptional repressors, antisense molecules, ribozymes, small inhibitory nucleic acid sequences, for example but not limited to RNAi, shRNAi, siRNA, micro-RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. The agent may be applied to the media, where it contacts the cell and induces its effects. Alternatively, the agent may be intracellular within the cell as a result of introduction of the nucleic acid sequence into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In some embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

The term “antibody,” as used herein, refers to an immunoglobulin molecule which specifically binds with an antigen. The term “antibody fragment” refers to a portion of an intact antibody and refers to the antigenic determining variable regions of an intact antibody.

The term “culture conditions” or a “culture medium” or “medium” can be used interchangeably and refers to a medium for culturing cells containing nutrients that maintain cell viability and support cell expansion and maintenance stage or the cell differentiation stage. The cell culture medium, in addition to the embodiments disclosed herein, may contain any of the following in an appropriate combination: salt(s), buffer(s), amino acids, glucose or other sugar(s), antibiotics, serum or serum replacement, and other components such as peptide growth factors, etc. The appropriate cell culture media, for a particular cell type, is known to those skilled in the art.

As used herein, the terms “treat,” treating,” “treatment,” and the like refer to reducing or ameliorating a disorder and/or symptom associated therewith. It will be appreciated that, although not precluded, treating a disorder or condition does not require that the disorder, condition or symptoms associated therewith be eliminated.

The term “comprises,” “comprising,” “containing” and “having” and the like can have the meaning ascribed to them in U.S. patent law and can mean “includes,” “including,” and the like (e.g., a composition “comprising” X may consist exclusively of X or may include something additional, e.g., X+Y); “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Specifically stated or obvious from context, as used herein, the term “or” is understood to be inclusive. Unless specifically stated or obvious from context, as used herein, the terms “a”, “an”, and “the” are understood to be singular or plural.

The word “substantially” does not exclude “completely” e.g., a composition which is “substantially free” from Y may be completely free from Y. Where necessary, the word “substantially” may be omitted from the definition of the disclosure.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The term “linker” as used herein, refers to “bifunctional crosslinking agent,” “bifunctional linker” or “crosslinking agents” refers to modifying agents that possess two reactive groups; one of which is capable of reacting with a cell binding agent while the other one reacts with the cytotoxic compound to link the two moieties together. Such bifunctional crosslinkers are well known in the art (see, for example, Islam and Dent in Bioconjugation chapter 5, p 218-363, Groves Dictionaries Inc. New York, 1999). A “linker,” “linker moiety,” or “linking group” as defined herein also refers to a moiety that connects two groups, such as a cell binding agent and a cytotoxic compound, together. Typically, the linker is substantially inert under conditions for which the two groups it is connecting are linked. A bifunctional crosslinking agent may comprise two reactive groups, one at each ends of a linker moiety, such that one reactive group can be first reacted with the cytotoxic compound to provide a compound bearing the linker moiety and a second reactive group, which can then react with a cell binding agent. Alternatively, one end of the bifunctional crosslinking agent can be first reacted with the cell binding agent to provide a cell binding agent bearing a linker moiety and a second reactive group, which can then react with a cytotoxic compound. The linking moiety may contain a chemical bond that allows for the release of the cytotoxic moiety at a specific site. Suitable chemical bonds are well known in the art and include disulfide bonds, thioether bonds, acid labile bonds, photolabile bonds, peptidase labile bonds and esterase labile bonds (see for example U.S. Pat. Nos. 5,208,020; 5,475,092; 6,441,163; 6,716,821; 6,913,748; 7,276,497; 7,276,499; 7,368,565; 7,388,026 and 7,414,073). Preferred are disulfide bonds, thioether and peptidase labile bonds. Other linkers that can be used in the present disclosure include non-cleavable linkers, such as those described in are described in detail in U.S. Publication No. 20050169933 or charged linkers or hydrophilic linkers and are described in U.S. Publication Nos. 2009/0274713 and US 2010/01293140; and PCT Patent Publication No. WO 2009/134976, each of which is expressly incorporated herein by reference, each of which is expressly incorporated herein by reference. A “Linker” (L) is a bifunctional or multifunctional moiety that can be used to link one or more drug moieties (d) to a platelet variant to form an platelet variant bioconjugate of formula platelet variant-L-C. In some embodiments, platelet variant-drug conjugates can be prepared using a linker having reactive functionalities for covalently attaching to the drug and to the platelet variant. for example, in some embodiments, a cysteine thiol of a platelet variant receptor (e.g., CD68, CD36, CD42b, lactadherin, etc.) can form a bond with a reactive functional group of a linker or a drug-linker intermediate to make a platelet variant-bioconjugate.

“Cleavable” as used herein refers to a linker or linker component that connects two moieties by covalent connections but breaks down to sever the covalent connection between the moieties under physiologically relevant conditions, typically a cleavable linker is severed in vivo more rapidly in an intracellular environment than when outside a cell, causing release of the payload to preferentially occur inside a targeted cell. Cleavage may be enzymatic or non-enzymatic, but generally releases a payload from an antibody without degrading the antibody. Cleavage may leave some portion of a linker or linker component attached to the payload, or it may release the payload without any residual part or component of the linker.

“Non-cleavable” as used herein refers to a linker or linker component that is not especially susceptible to breaking down under physiological conditions, e.g., it is at least as stable as the platelet variant receptor proteins. Such linkers are sometimes referred to as “stable”, meaning they are sufficiently resistant to degradation to keep the payload connected to the platelet variant receptor until platelet variant is itself at least partially degraded, i.e., the degradation of platelet variant precedes cleavage of the linker in vivo.

“Bioconjugation” refers to conjugating platelet variants or derivatives thereof to a cytotoxic agent with or without the use of a linker. Bioconjugation techniques are well known to one of skill and the art and can be found, for example, in Bioconjugate Techniques, 3rd Edition (2013) by Greg T. Hermanson (ISBN 978-0-12-382239-0: Academic Press). “Bioconjugate Techniques” besides being a complete textbook and protocols-manual for biomolecular crosslinking, it is also an exhaustive and robust reference for conjugation strategies.

The term “amino acid” refers to naturally occurring and synthetic amino acids, as well as amino acid analogs and amino acid mimetics that function in a manner like the naturally occurring amino acids. Naturally occurring amino acids are those encoded by the genetic code, as well as those amino acids that are later modified, e.g., hydroxyproline, gamma-carboxyglutamate, selinocystiene and O-phosphoserine. Amino acid analogs may refer to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but functions in a manner like a naturally occurring amino acid. The synthetically modified forms include, but are not limited to, amino acids having side chains shortened or lengthened by up to two carbon atoms, amino acids comprising optionally substituted aryl groups, and amino acids comprised halogenated groups, preferably halogenated alkyl and aryl groups and also N substituted amino acids e.g. N-methyl-alanine. An amino acid or peptide can be attached to a linker/spacer or a cytotoxic agent through the terminal amine or terminal carboxylic acid of the amino acid or peptide. The amino acid can also be attached to a linker/spacer or a cytotoxic agent through a side chain reactive group, such as but not restricted to the thiol group of cysteine, the epsilon amine of lysine or the side chain hydroxyls of serine or threonine.

Amino acids and peptides may be protected by blocking groups. A blocking group is an atom or a chemical moiety that protects the N-terminus of an amino acid or a peptide from undesired reactions and can be used during the synthesis of a drug-ligand conjugate. It should remain attached to the N-terminus throughout the synthesis and may be removed after completion of synthesis of the drug conjugate by chemical or other conditions that selectively achieve its removal. The blocking groups suitable for N-terminus protection are well known in the art of peptide chemistry. Exemplary blocking groups include, but are not limited to, methyl esters, tert-butyl esters, 9-fluorenylmethyl carbamate (Fmoc) and carbobenzoxy (Cbz).

Donor derived platelets (i.e., bone marrow derived platelets) are always constrained by their inherent limitation, i.e., their source, because their source is always dependent on a volunteer donor. Absence a donor, there are no bone marrow derived platelets. Given the critical roles played by platelets in hemostasis and thrombosis, the maintenance of vascular integrity, the development of the lymphatic system, the innate immune response, the pathophysiology of inflammation and tumor growth and metastasis, it becomes imperative to develop methodologies to enhance mature megakaryocytes production from progenitor cells as well as develop methodologies for generating platelets or platelet variants or derivatives thereof that are readily available to treat various diseases and disorders.

Thus, in some embodiments, the present disclosure is directed to an improved method for generating megakaryocytes from CD34+ progenitor stem cells, which comprises at least two stages, a stage zero (0) and a stage (I). The stage zero (0) comprises an expansion and maintenance stage of the CD34+ progenitor stem cells for a period sufficient to expand and maintain the CD34+ progenitor stem cells. Such period could last between a few hours to up to 10 days or between 1 day and 9 days or between 1 day and 8 days or between 1 day and 7 days or between 1 day 6 days or between 1 day to 5 days or between 1 day and 4 days or between 1 day and 3 days or between 1 day to 2 days. For example, stage zero could last for 5 hours, 10 hours, 15 hours, 20 hours, 25 hours, 30 hours, 35 hours, 40 hours, 45 hours, 48 hours or 50 hours. In some embodiments, stage zero could last between 0-5 hours, 5-10 hours, 10-15 hours, 15-20 hours, 20-25 hours, 25-30 hours, 30-35 hours, 35-40 hours, 40-45 hours, 45-48 hours or between 2 to 4 days or between 4 to 6 days or between 6 to 8 days or between 8 to 10 days or for 2 weeks or 3 weeks.

Upon completion of stage (0), i.e., the expansion and maintenance stage of the CD34+ progenitor stem cells, the progenitor cells enter stage (I), which comprises a differentiation stage of the CD34+ progenitor stem cells after completion of stage zero. The differentiation stage (i.e., stage I) comprises differentiating the CD34+ progenitor stem cells, after completion of step (i), for a period of time sufficient to generate substantially matured megakaryocytes. Such differentiation stage, i.e., stage (I) could last between a few hours to up to 10 days after the completion of stage zero. For example, stage (I) can last for 1 day or 15 days or 1 to 14 or 1 to 13 days or 1 to 12 days or 1 to 11 days or 1 to 10 days or 1 to 9 days or 1 to 8 days or 1 to 7 days or 1 to 6 days or 1 to 5 days or 1 to 4 days or 1 to 3 days or 1 to 2 days after the completion of stage (0). In some embodiments, the stage (I) could last between 1-2 days, 2-3 days, 3-4 days, 4-5 days, 5-6 days, 7-8 days, 8-9 days or 9-10 days, 10-11 days, 11 to 12 day, 12-13 days, 13 to 14 days or for 3 weeks to 4 weeks after the completion of stage (0).

In some embodiments, each stage, i.e., stage (0) and stage (I), each require independent culture constituents, which may or may not overlap with each other. For example, in stage (0), i.e., the expansion and maintenance stage, CD34+ progenitor cells are cultured in an expansion medium (EXM) comprising SFEM (STEMCELL Technologies), PEN/Strep (Thermo Fisher Scientific), glutamine (Thermo Fisher Scientific), lipids (Sigma), rhTPO, Flt-3, and SCF (PeproTech), each in concentrations sufficient to facilitate the expansion of the CD34+ progenitor cells. Each constituent, depending on the culture need, may be added concurrently, constitutively or consecutively. For example, the EXM may be lacking in Flt-3, which may be added later on.

Whereas, in stage (I), the EXM is removed and replaced with a differentiation medium (DM) comprising SFEM, low density lipids (STEMCELL Technologies), SCF (PeproTech), rhTPO (STEMCELL Technologies), IL-9 (PeproTech), and IL-6 (PeproTech), each in concentrations sufficient to facilitate the differentiation of the CD34+ progenitor cells. Each constituent, depending on the culture need, may be added concurrently, constitutively or consecutively.

In some embodiments, MKs can be derived from CD34+ pluripotent stem cells, including but not limited to, embryonic stem cells (ESCs) (e.g. human embryonic stem cells). ESCs are pluripotent stem cells derived from the inner cell mass of an early-stage preimplantation embryo called a blastocyst.

In some embodiments, MKs can be derived from hematopoietic stem cells, including but not limited, to CD34+ umbilical cord blood stem cells (UCB cells) (e.g. human CD34+ umbilical cord blood stem cells), CD34+ mobilized peripheral blood cells (MPB cells) (e.g. CD34+ human mobilized peripheral blood), or CD34+ bone marrow cells. UCB cells are multipotent stem cells derived from blood that remains in the placenta and the attached umbilical cord after childbirth. MPB cells are multipotent stem cells derived from volunteers whose stem cells are mobilized into the bloodstream by administration of G-CSF or similar agent.

In some embodiments, MKs can be derived from other stem cell types, including but not limited to mesenchymal stem cells (MSC) (such as, adipose-derived mesenchymal stem cells (AdMSC)) or mesenchymal stem from other sources.

AdMSCs are derived from white adipose tissue, which is derived from the mesoderm during embryonic development and is present in every mammalian species, located throughout the body. Due to their wide availability and ability to differentiate into other tissue types of the mesoderm-including bone, cartilage, muscle, and adipose-ASCs may serve a wide variety of applications.

In some embodiments, the CD34+ stem cell cultures are maintained independently of embryonic fibroblast feeder cells and/or animal serum that can potentially be contaminated with xenogeneic pathogens and increase the risk for an immunogenic reaction in humans. Therefore, serum-free, feeder-cell free alternatives are utilized to avoid the introduction of animal products into the preMKs and MKs derived according to the instant methods to ensure safe and animal product-free conditions and products.

In some embodiments, prior to subjecting the CD34+ progenitor cells to culturing in stage (0) or stage (I), isolated population of the CD34+ progenitor cells are genetically edited to induce or alter the expression of one of more MK-specific promoters to facilitate the progenitor cell differentiation along the megakaryocyte lineage. There are several megakaryocyte-specific gene promoters that can be utilized of this purpose including: members of the glycoprotein (GP) GPIBA-GPIX-GPV complex, ITGA2B (aka, integrin αIIb, GPIIb), GPVI, c-mpl, and platelet factor 4 (PF4). These promoters bind GATA-1, Ets (Fli-1), and FOG-1 factors that induce transcription in early and mid-stages of megakaryocytopoiesis. For example, PF4 is expressed at high levels during megakaryocytopoiesis and stored within platelet α-granules. Its gene promoter and distal regulatory regions are well characterized and have been shown to be useful for controlling megakaryocyte-specific transgene expression. These gene promoters could potentially be edited to expedite the expression of megakaryocyte-specific expression within the confines of the progenitor cells. Genetic editing can be accomplished by the well-established gene editing tool of the CRISPR-Cas systems. (Moon, S. B., Kim, D. Y., Ko, J. et al. Recent advances in the CRISPR genome editing tool set. Exp Mol Med 51, 1-11 (2019), incorporated herein in its entirety by reference).

In some embodiments, megakaryocyte-specific gene promoters, such as but not limited to, members of the glycoprotein (GP) GPIBA-GPIX-GPV complex, ITGA2B (aka, integrin αIIb, GPIIb), GPVI, c-mpl, and platelet factor 4 (PF4) could potentially direct transgene transcription that induce transcription in early and mid-stages of megakaryocytopoiesis. For example, PF4 is expressed at high levels during megakaryocytopoiesis and stored within platelet α-granules. Its gene promoter and distal regulatory regions are well characterized and have been shown to be useful for controlling megakaryocyte-specific transgene expression. Likewise, megakaryocyte-specific gene promoters, such as but not limited to, GP1BA, ITGA2B, and PF4 can used within gene transfer vectors used for megakaryocyte modification because they have been shown to consistently drive moderate- to high-level protein expression preferentially within megakaryocytes. These gene promoters could potentially be used to drive megakaryocyte-specific transgene expression within the confines of a variety of gene transfer vectors that have been characterized for their ability to efficiently and safely express the transgene product including in recombinant lentiviral, adenoviral, and adeno-associated viral vectors as well as plasmid DNA.

In some embodiments, the CD34+ progenitor cells (e.g., peripheral blood (PB) cells or Megakaryocytes derived therefrom), producing platelets, during stage (I) or after the completion of stage (I) are processed through a bioreactor to produce platelet or novel variants thereof referred to herein as platelets variants.

In some embodiments, the platelet variants or derivatives thereof derived from the progenitor cells of the present disclosure can be generated in a device or a system that supports a biologically active environment e.g., bioreactors or fluidic devices. Bioreactors or fluidic devices could include, but is not limited to, shear stress, mechanical strain and pulsed electromagnetic field bioreactors, large-scale stirred tank bioreactors, automated bioreactors, rotating wall bioreactors (RWBs), and rocking motions as seen with wave bioreactors, organ-on-chip bioreactors. Other bioreactor configurations that enable continuous, perfusion operation such as packed bed bioreactors (PBBs), fluidized bed bioreactors (FBBs), or PBBs or FBBs including the use of microcarriers, CultiBag bioreactors, and membrane bioreactors such as hollow fiber bioreactors (HFBs) are also contemplated for generating the platelet variants or derivatives thereof of the present disclosure. Operation of the bioreactors may require coupling with an internal or external cell retention device on a recycle line, by centrifugation, sedimentation, ultrasonic separation or microfiltration with spin-filters, alternating tangential flow (ATF) filtration or tangential flow filtration (TFF) or in vivo bioreactors, which are a pocket within the body into which biomaterials (e.g., platelet variants or their derivatives or the progenitor cells form which they are derived from) are implanted at a site in need thereof and incubated for an extended period of time. Within these pockets (for example, bone tissue or muscle flap etc.), the grafts harness the regenerative capacity of the body to recover from a disease or an injury. Non-limiting examples of bioreactors are described, for example, in the co-filed application titled: Simultaneous Welding of Three Components To Form a Bioreactor or Filter Structure (PCT/US2021/019660) or elsewhere, for example tools and technologies (e.g., bioreactors or fluidic devices) disclosed in U.S. Pat. Nos. 9,795,965; 10,343,163; 9,763,984; 9,993,503; and 10,426,799; U.S. Patent Publication No. 20180334652; PCT Patent Application Nos. PCT/US2018/021354; PCT/US2019/012437; PCT/US2019/040021; and U.S. patent application Ser. No. 16/730,603, each of which is incorporated herein in their entirely by reference. Bioreactors or microfluidic devices known or unknown that can routinely generate the platelet variants or derivatives are also contemplated for use in the present disclosure. For example, non-naturally occurring platelet variants or derivatives thereof can be generated in a bioreactor, which mimics the in vivo environment for the endogenous platelet generation in humans.

Given that the platelets are made in a bioreactor, the platelets, in some embodiments, are generated ex-vivo in an environment that differs from the in-vivo environment. Hence, the platelets derived from the CD34+ progenitor cells are platelets as well as variants of donor platelets. The bioreactor processed variant platelets are unique as compared to and bear unique structural characteristics by which they can be distinguished from bone morrow derived platelets. While structurally different, the platelet variants retain many of functional indices of bone marrow derived platelets, which makes the CD34+ platelet variants or derivatives thereof a readily available substitute for the donor platelets. Thus, the CD34+ platelet variants or derivatives thereof provide unique utility as a replacement for donor platelets or for treating diseases or disorders where bone marrow platelets play a role but are in short supply or are defective in their physiological roles.

In some embodiments, the platelet variants of the present disclosure are structurally characterized as:

CD62p>average3.65% as compared to the platelets derived from the same source, i.e., CD34+ progenitor cell derived platelet cells.

CD62p>average5% as compared to the platelets derived from the same source, i.e., CD34+ progenitor cell derived platelet cells.

CD62p>average7% as compared to the platelets derived from the same source, i.e., CD34+ progenitor cell derived platelet cells.

CD62p>average10% as compared to the platelets derived from the same source, i.e., CD34+ progenitor cell derived platelet cells.

CD62p>averagel5% as compared to the platelets derived from the same source, i.e., CD34+ progenitor cell derived platelet cells.

CD42a<average100% as compared to the platelets derived from the same source, i.e., CD34+ progenitor cell derived platelet cells.

CD42a<average95% as compared to the platelets derived from the same source i.e., CD34+ progenitor cell derived platelet cells.

CD42a<average90% as compared to the platelets derived from the same source i.e., CD34+ progenitor cell derived platelet cells.

CD42a<average85% as compared to the platelets derived from the same source i.e., CD34+ progenitor cell derived platelet cells.

CD42a<average80% as compared to the platelets derived from the same source i.e., CD34+ progenitor cell derived platelet cells.

Calcein<average99% as compared to the platelets derived from the same source i.e., CD34+ progenitor cell derived platelet cells.

Calcein<average95% as compared to the platelets derived from the same source i.e., CD34+ progenitor cell derived platelet cells.

Calcein<average93% as compared to the platelets derived from the same source i.e., CD34+ progenitor cell derived platelet cells.

Calcein<average90% as compared to the platelets derived from the same source i.e., CD34+ progenitor cell derived platelet cells.

Calcein<average87% as compared to the platelets derived from the same source i.e., CD34+ progenitor cell derived platelet cells.

Calcein<average84% as compared to the platelets derived from the same source i.e., CD34+ progenitor cell derived platelet cells.

In some embodiments, the platelet-producing CD34+ progenitor cells (e.g., peripheral blood cells or Megakaryocytes derived therefrom) are genetically engineered to produce platelets or variants thereof that deliver cytotoxic agents, such as but not limited to antibodies, cytokines or growth factors. The CD34+ progenitor cells (e.g., CD34+ peripheral blood cells or Megakaryocytes derived therefrom) that produce the platelets or variant platelets, or the platelets or the platelet variants, are genetically engineered by introducing into an isolated population of CD34+ progenitor cells or the platelet or the variant platelet population an exogenous nucleic acid. In some embodiments, the nucleic of the present disclosure, i.e., a nucleic acid encoding a protein of interest is operably linked to a regulatory element, can be stably inserted into isolated population of CD34+ progenitor cells (e.g., CD34+ peripheral blood cells or megakaryocytes derived therefrom) or the platelet variants derived therefrom as naked DNA or RNA or more commonly as part of a vector to facilitate manipulation of the nucleic acid. As used herein, the term nucleic acid refers to a nucleic acid molecule (e.g., encoding one or more proteins), which is inserted by artifice into a cell and is stably integrated into the chromosomal genome of the cell or is stably maintained as an episome.

Nucleic acids can be introduced into the isolated population of CD34+ progenitor cells (e.g., CD34+ peripheral blood cells or megakaryocytes derived therefrom) or the platelet variants derived therefrom by means of a viral vector, such as but not limited to adenoviral vectors, adeno-associated viral vectors, herpes simplex viral vectors, vaccinia viral vectors, baculoviral vectors or retroviral vectors. There are many retroviral based vectors. For the present application, the term “retrovirus” includes: murine leukemia virus (MLV), human immunodeficiency virus (HIV), equine infectious anemia virus (EIAV), mouse mammary tumor virus (MMTV), Rous sarcoma virus (RSV), Fujinami sarcoma virus (Fussy), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), adenoviral vectors, adeno-associated virus (AAV), and Avian erythroblastosis virus (AEV) and all other retroviridiae including lentiviruses.

### General Structure of The Vectors

Lentiviral vectors are at the forefront of gene delivery systems for research and clinical applications. These vectors can efficiently transduce nondividing and dividing cells, to insert large genetic segment in the host chromatin, and to sustain stable long-term transgene expression. Like other retroviruses, lentiviruses have gag, pol and env genes flanked by two LTR (Long Terminal Repeat) sequences. Each of these genes encodes for numerous proteins which are initially expressed in the form of a single precursor polypeptide. The gag gene encodes for the internal structure proteins (capsids and nucleocapsid). The pol gene encodes for inverse transcriptase, integrase and protease. The env gene encodes for viral envelope glycoprotein. Furthermore, the lentivirus genome contains a cis-acting RRE (Rev Responsive Element) element responsible for exporting out of the nucleus of the viral genomic RNA which will be packaged. The LTR 5′ and 3′ sequences serve to promote the transcription and polyadenylation of the viral RNAs. The LTR contains all the other cis-acting sequences necessary for viral replication. Sequences necessary for the inverse transcription of the genome (linkage site of the RNAt primer) and for the encapsidation of viral RNA in particles (T site) are adjacent to the LTR 5′. If the sequences necessary for encapsidation (or for packaging retroviral RNA in the infectious virions) are absent from the viral genome, genomic RNA will not be actively packaged. Furthermore, the lentiviral genome comprises accessory genes such as vif, vpr, vpu, nef, TAT, REV etc. The construction of lentiviral vectors for gene transfer applications has been described, for example, in patents U.S. Pat. No. 5,665,577; EP 386 882; U.S. Pat. Nos. 5,981,276 and 6,013,516; or in PCT Patent Publication Nos. WO99/58701 and WO02/097104, incorporated herein by reference in their entireties. These vectors include a defective lentiviral genome, i.e. in which at least one of the gags, pol and env genes has been inactivated or deleted.

Lentivirus experiments can be performed using lentivirus vectors known to one of skill in the art. As a non-limiting example, lentivirus vectors such as but not limited to GCMV-MCS-IRES-eGFP and GCMV-MCS-IRES-dsRed can be used to deliver a transgene of interest. Both vectors are HIV1 strains that lack the structural viral genes gag, pol, env, rev, tat, vpr, vif, vpu, and nef. In addition, there is a partial deletion of the promoter/enhancer sequences within the 3′ LTR that renders the 5′ LTR/promoter self-inactivating following integration. The genes provided in trans for both vectors are the structural viral proteins Gag, Pol, Rev, and Tat (via plasmid Delta 8.9) and the envelope protein VSV-G. These plasmids are introduced into the CD34+ platelet variants by co-transfection, and transiently express the different viral proteins required to generate viral particles. The potential for generating wild type or pathogenic lentivirus is extremely low because it would require multiple recombination events amongst three plasmids. In addition, the virulence factors (vpr, vif, vpu, and nef) have been completely deleted from both vectors.

In some embodiments, the isolated population of CD34+ progenitor cells (e.g., CD34+ peripheral blood cells or megakaryocytes derived therefrom) are genetically engineered by introducing into an isolated population of CD34+ progenitor cells (e.g., CD34+ peripheral blood cells or megakaryocytes derived therefrom) a first transgene comprising an inducible promoter and nucleotide sequences encoding one or more exogenous proteins for their transcription under the control of the inducible promoter. Alternatively, a second transgene is introduced into the same platelet population or their progenitor cells, the second transgene comprising a constitutive promoter and a nucleotide sequence encoding a transcription factor for the constitutive expression of the transcription factor under the control of the constitutive promoter, the transcription factor specific for binding to the inducible promoter in the first transgene thereby inducing transcription of the proteins from the coding sequences in the first transgene.

In the case of an antibody or a fragment thereof that is encoded by a transgene or an antibody or a fragment thereof is conjugated to the platelet variants, the antibody or the fragment thereof is preferably a cell binding agent, i.e., the antibody or the fragment thereof binds to one or more receptors or ligands or antigens or to any other binding element on a cell for which the antibody is specific for commonly known to one of skill in the art. For example, the antibody anti-CTLA4 is a hIgG1 antibody that binds specifically to the CTLA4 receptors and can be used if the target T cells expressing CTLA4.

The cell-binding agent may be any compound that can bind a cell, either in a specific or non-specific manner. Generally, these can be antibodies (especially monoclonal antibodies and antibody fragments), interferons, lymphokines, hormones, growth factors (e.g., HGF), vitamins, nutrient-transport molecules (such as transferrin), blood-coagulation factor VIIa, or any other cell-binding molecule or substance.

In some embodiments, the exogenous genetic material may be selected from, but not limited to, siRNA, shRNA, cDNA, DNA, in one or separate vectors with independent inducible (e.g., Tetracycline (Tet) Inducible Expression) or constitutive promoters or a combination thereof. The transgene of the present disclosure, i.e., a nucleic acid encoding a protein of interest is operably linked to a constitutive or inducible regulatory elements that can be stably inserted into the CD34+ progenitor cells (e.g., CD34+ peripheral blood cells or megakaryocytes derived therefrom) or the platelet variants derived therefrom naked DNA or more commonly as part of a vector to facilitate manipulation of the transgene. Viral vectors are well known to skill of the art and deposits of such vectors are commercially available at https://www.addgene.org/. Non-limiting examples of genetically engineering platelet variants are shown in a co-pending U.S. patent application Ser. No. 17/213,552, incorporated herein by reference in its entirety.

Infusion of Cytotoxic Agents into the Platelets or the Platelet Variants

This novel strategy takes advantage of the platelets (e.g., CD34+-derived platelets) or the platelet variants' properties, such as, flexible morphology to offer a unique opportunity to maximize therapeutic outcomes as well as minimizing side effects. This novel strategy also takes advantage of the platelet's or the platelet variant's unique property to store secretory granules, which remain stored in the platelets or the platelet variants or derivatives thereof until platelet variants or derivatives thereof trigger their release.

Thus, some embodiments take the advantage of the cell membrane permeability of the platelets or the platelet variants or derivatives thereof in hypotonic solution, which enables the entrapment of drugs, biomacromolecules, and nanoparticles into the cavities of the platelets or platelet variants or derivatives thereof, generally reserved for storing platelets or platelet variant-derived secretory granules. The principle for infusion of cytotoxic agents into the platelets or platelet variants or derivatives thereof is because the absence of a superfluous membrane on the platelets or platelet variants allows the platelets or the platelet variants to accommodate additional volume by changing the shape, for example, from biconcave to spherical. Several hypotonic hemolysis techniques have been generated, such as hypotonic dialysis, hypotonic dilution, and hypotonic pre-swelling. Hypotonic dialysis is predominantly applied in encapsulating enzymes, proteins, and contrast agents due to its relative ease of use, ability to preserving characteristics of the platelet variants or their derivatives and high encapsulation rate. In the process, platelets or platelet variants or their derivatives may be prepared in a dialysis tube and immersed in a hypotonic buffer for a few hours under gentle stirring. Nucleic acids (e.g., RNAs or DNAs) or proteins (e.g., antibodies or fragments thereof), as therapeutic agents, may loaded into the platelets or the platelet variants via hypotonic dialysis and achieve between 20% to 90% encapsulation after 2-28 hours incubation. Nucleic acid platelets or the platelet variants or derivatives thereof may undergo opsonization with ZnCl2 and bis-sulfosuccynimidil-suberate treatment, and then specifically could be used to target a cell or a tissue, such as a tumor cell or T-cells or macrophages, at a second location. In this manner nucleic acid or proteins may effectively be delivered and result in production of enzymatic activities or physiological reactions to inhibit protein expression, for example the platelets or the platelet variants or derivatives thereof may induce nitric oxide synthesis thereby blocking recruitment of bone marrow derived platelets at tumor cites in a tumor microenvironment thereby preventing tumor metastasis. It is well known that upon tumor cell arrival in the blood, tumor cells immediately activate platelets to form a permissive microenvironment. Platelets protect tumor cells from shear forces and assault of NK cells, recruit myeloid cells by secretion of chemokines, and mediate an arrest of the tumor cell platelet embolus at the vascular wall. Subsequently, platelet-derived growth factors confer a mesenchymal-like phenotype to tumor cells and open the capillary endothelium to expedite extravasation in distant organs. Finally, platelet-secreted growth factors stimulate tumor cell proliferation to micrometastatic foci. Thus, in some embodiments the platelets or the platelet variants or derivatives of the present disclosure could act as a decoy to fool metastasizing tumor cells into communicating with payload bearing platelets or the platelet variants rather than endogenous platelets thereby limiting the tumor metastasizing role played by endogenous platelets.

### Receptors in Platelets and Platelet Variants

The CD34+ derived platelets or the platelet variant or derivatives thereof may share one or more of the following receptors, whether cell surface or transmembrane, inclusive of, but not limited to, P2Y1, P2Y12, PAR1, PAR4, Tpa, PAF receptors, PGE2 receptor (EP3), Lysophosphatidic acid receptor, Chemokine receptors, V1a vasopressin receptor, A2a adenosine receptor, b2 adrenergic receptor, Serotonin receptor, Dopamine receptor, P2X1, c-Mp1, Insulin receptor, PDGF receptor, Leptin receptor, GPVI, CD148, CLEC-2, Eph receptor, Ax1/Tyro3/Mer, P-selectin, TSSC6, CD151, CD36, TLT-1, PEAR1, VPAC1, PECAM-1, G6B-b, PGI2 receptor (IP), PGD2 receptor, PGE2 receptor (EP4), GPIb-IX-V complex or a modified version thereof.

### Receptor Families in Platelets and Platelet Variants or Derivatives Thereof

In some embodiments, one of skill in the art may easily replace one receptor with another belonging to same or different families of platelet variant receptors. A skilled artisan can pick one or more receptors from the Leucine-rich repeat family, Ig superfamily, Integrins, Tyrosine phosphatase receptor, C-type lectin receptor, G protein-coupled receptors, Ion channel, Tyrosine kinase receptor, Cytokine, C-type lectin receptor family, tetraspanins, Class B scavenger receptor, Multiple EGF-like domain protein, transmembrane 4 superfamily, as these families are generally inclusive of receptors on the platelet variants.

### Ligands

Several ligands specifically bind to platelet variants' receptors. For example, Willebrand factor (VWF) interacts with the platelet variants' receptors, glycoprotein (GP) Ib-IX-V and αIIbβ3 integrin, to promote primary platelet adhesion and aggregation following vessel injury. The ability VWF to bind to platelet variant receptor GPIb-IX-V provides a target for the treatment of diseases related to arterial and venous pathological thrombosis. Likewise, CD36 receptors on the platelet variants recognize at least three classes of ligand: modified phospholipids, a subset of proteins containing a structural domain termed the thrombospondin type I repeat (TSR), and free fatty acids. Studies have shown that loss of CD36 confers substantial protection against atherosclerosis. In contrast, CD36-mediated anti-angiogenesis is caused by its ability to activate a specific signaling cascade that results in diversion of a proangiogenic response to an apoptotic response. Thus, the CD36 receptors in the platelet variants can be genetically manipulated or chemically modified to influence the CD36 receptor's interaction with the ligands (e.g., TSP1, oxLDL, VLDL, oxPL). Here a patient suffering from a CD36 related disorder can benefit by manipulating the CD36 receptor or the ligands that bind to it to provide a therapeutic relief (e.g., protection from atherosclerosis).

Ligands that can be bioconjugated to a cytotoxic agent, such proteins, polypeptides, nucleic acid molecules or small molecule drugs are inclusive of but limited to, vWf, thrombin, FXI, FXII, P-selectin, HK, Mac-1, TSP-1, Collagen, laminin, Fibronectin, Vitronectin, fibrinogen, osteopontin, fibrin, TSP-1, Podoplanin, ADP, Thrombin, Thromboxane, 1-O-alkyl-2-acetyl-sn-glycero-3-phosphocholine, PGE2, Lysophosphatidic acid, Chemokines, Vasopressin, Adenosine, Epinephrine, Serotonin (5-hydroxytryptamin), Dopamine, ATP, TPO, Insulin, PDGF, Leptin, Ephrin, Gas-6, PSGL-1, GPIb, TF, TSP1, oxLDL, VLDL, oxPL, collagen type V, Fibrinogen, PACAP, PECAM-1, collagen, glycosaminoglycans, PGI2, PGD2.

Antigens and Antibodies for Use with the Platelet Variants

Antigens and antibodies or fragments thereof, being proteinaceous in nature, provide several advantages for platelet-based therapeutic payload delivery. For example, platelets or the platelet variants or the CD34+ progenitor cells making the platelets or platelet variants or derivatives thereof can be genetically engineered to produce antigens or antibodies or fragments thereof as disclosed herein. For conjugation of an antibody or fragments thereof to the platelets or the platelet variants or derivatives thereof, one or more amino acids of an antibody or a fragment thereof can be directly bioconjugated to cell surface or transmembrane receptor proteins or ligands, without the need of a chemical linking agent, of the platelet or the platelet variants or derivatives thereof. Further, antibodies or fragments thereof are capable of binding to antigens on the platelets or the platelet variant receptors or ligands. Lastly, antibodies can be attached to the platelets or the platelet variants or derivatives thereof via linkers, as discussed in the foregoing. In some embodiments, the platelets or the platelet variants or the CD34+ progenitor cells from which the platelets and the platelet variants are derived from can be genetically engineered in a lentivirus-based vector to produce ipilimumab, a monoclonal antibody that works to activate the immune system by targeting CTLA-4, a protein receptor that downregulates the immune system. In some embodiments, ipilimumab may be bioconjugated to the platelets or the platelet variant receptor proteins through protein-protein conjugation or it may be conjugated via maleimide cross-linking reaction to free thiols which allows for stable conjugation to the platelets or the platelet variant surface protein having a modified thiol group or via a platelet variant cell surface or a modified platelet variant cell surface or a transmembrane protein.

Exemplary antigens that may be genetically engineered into CD34+ progenitor cells from which the platelets or the platelet variants or derivatives thereof are derived from, for example, in a lentivirus based vector, or are bioconjugated to the platelets or the platelet variants or derivatives thereof include molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor vmc, factor IX, tissue factor (TF), and von Willebrand factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin, such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-.beta.; platelet-derived growth factor (PDGF); fibroblast growth factor such as aFGF and bFGF; fibroblast growth factor receptor 2 (FGFR2), epidermal growth factor (EGF); transforming growth factor (TGF) such as TGF-alpha and TGF-beta, including TGF-beta1, TGF-beta2, TGF-beta3, TGF-beta4, or TGF-beta5; bone morphogenetic protein (BMP), including BMP1, BMP6, BMP7, and BMP-receptor 2; insulin-like growth factor-I and -II (IGF-I and IGF-II); des(1-3)-IGF-I (brain IGF-I), insulin-like growth factor binding proteins, EpCAM, GD3, FLT3, PSMA, PSCA, MUC1, MUC16, STEAP, CEA, TENB2, EphA receptors, EphB receptors, folate receptor, FOLR1, mesothelin, cripto, alphavbeta6, integrins, VEGF, VEGFR, EGFR, transferrin receptor, IRTA1, IRTA2, IRTA3, IRTA4, IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80. CD81, CD103, CD105, CD134, CD137, CD138, CD152, IFN gamma TNF alpha, IFN alpha, GM-CSF, IL-3 or an antibody which binds to one or more tumor-associated antigens or cell-surface receptors; erythropoietin; osteoinductive factors; immunotoxins; a bone morphogenetic protein (BMP); an interferon, such as interferon-alpha, -beta, and -gamma; colony stimulating factors (CSFs), e.g., M-CSF, GM-CSF, and G-CSF; interleukins (ILs), e.g., IL-2, IL-6, IL-12, IL-23, IL-12/23 p40, IL-17, IL-15, IL-21, IL-1a, IL-1b, IL-18, IL-8, IL-4, IL-3, and IL-5; superoxide dismutase; T-cell receptors; surface membrane proteins; decay accelerating factor; viral antigen such as, for example, a portion of the HIV envelope; transport proteins; homing receptors; addressing; regulatory proteins; integrins, such as CD11a, CD11b, CD11c, CD18, an ICAM, VLA-4 and VCAM; a tumor associated antigen such as HER2, HER3 or HER4 receptor; endoglin, c-Met, c-kit, 1GF1R, PSGR, NGEP, PSMA, PSCA, LGRS, B7H4, TAG72 (tumor-associated glycoprotein 72) and fragments of any of the above-listed polypeptides.

Examples of antibodies that the platelets or the platelet variants or derivatives thereof can produce (for example, from genetically engineered CD34+ progenitor cells by using a lentivirus based vectors to express the exogenous protein of interest) or that can be linked to the platelets or the platelet variants or derivatives thereof include, but are not limited to, abciximab (Reopro), adalimumab (Humira, Amjevita), alefacept (Amevive), alemtuzumab (Campath), basiliximab (Simulect), belimumab (Benlysta), bezlotoxumab (Zinplava), canakinumab (Ilaris), certolizumab pegol (Cimzia), cetuximab (Erbitux), daclizumab (Zenapax, Zinbryta), denosumab (Prolia, Xgeva), efalizumab (Raptiva), golimumab (Simponi, Simponi Aria), inflectra (Remicade), ipilimumab (Yervoy), ixekizumab (Taltz), natalizumab (Tysabri), nivolumab (Opdivo), olaratumab (Lartruvo), omalizumab (Xolair), palivizumab (Synagis), panitumumab (Vectibix), pembrolizumab (Keytruda), rituximab (Rituxan), tocilizumab (Actemra), trastuzumab (Herceptin), secukinumab (Cosentyx), ranibizumab, abciximab, raxibacumab, caplacizumab, infliximab, bevacizumab, dabigatran, Idarucizumab, or ustekinumab (Stelara) or a combination thereof. Furthermore, the antibodies may be selected from anti-estrogen receptor antibody, anti-progesterone receptor antibody, anti-p53 antibody, anti-EGFR antibody, anti-cathepsin D antibody, anti-Bcl-2 antibody, anti-E-cadherin antibody, anti-CA125 antibody, anti-CA15-3 antibody, anti-CA19-9 antibody, anti-c-erbB-2 antibody, anti-P-glycoprotein antibody, anti-CEA antibody, anti-retinoblastoma protein antibody, anti-ras oncoprotein antibody, anti-Lewis X antibody, anti-Ki-67 antibody, anti-PCNA antibody, anti-CD3 antibody, anti-CD4 antibody, anti-CD5 antibody, anti-CD7 antibody, anti-CD8 antibody, anti-CD9/p24 antibody, anti-CD1-antibody, anti-CD11c antibody, anti-CD13 antibody, anti-CD14 antibody, anti-CD15 antibody, anti-CD19 antibody, anti-CD20 antibody, anti-CD22 antibody, anti-CD23 antibody, anti-CD30 antibody, anti-CD31 antibody, anti-CD33 antibody, anti-CD34+ antibody, anti-CD35 antibody, anti-CD38 antibody, anti-CD39 antibody, anti-CD41 antibody, anti-LCA/CD45 antibody, anti-CD45RO antibody, anti-CD45RA antibody, anti-CD71 antibody, anti-CD95/Fas antibody, anti-CD99 antibody, anti-CD100 antibody, anti-S-100 antibody, anti-CD106 antibody, anti-ubiquitin antibody, anti-c-myc antibody, anti-cytokeratin antibody, anti-lambda light chains antibody, anti-melanosomes antibody, anti-prostate specific antigen antibody, anti-tau antigen antibody, anti-fibrin antibody, anti-keratins antibody, and anti-Tn-antigen antibody.

Monoclonal antibody techniques allow to produce extremely specific cytotoxic agent in the form of specific monoclonal antibodies. Particularly well known in the art are techniques for creating monoclonal antibodies produced by immunizing mice, rats, hamsters or any other mammal with the antigen of interest such as the intact target cell, antigens isolated from the target cell, whole virus, attenuated whole virus, and viral proteins such as viral coat proteins. Sensitized human cells can also be used. Another method of creating monoclonal antibodies is the use of phage libraries of scFv (single chain variable region), specifically human scFv (see e.g., Griffiths et al.; U.S. Pat. Nos. 5,885,793 and 5,969,108; McCafferty et al.; PCT Patent Publication No. WO 92/01047; Liming et al.; and PCT Patent Publication No. WO 99/06587). In addition, resurfaced antibodies disclosed in U.S. Pat. No. 5,639,641 may also be used, as may chimeric antibodies and humanized antibodies. Selection of the appropriate cytotoxic agent is a matter of choice that depends upon the cell population that is to be targeted, but in general human monoclonal antibodies or fragments thereof are preferred if an appropriate one is available.

### Bioconjugates

In some embodiments, the platelets or the platelet variants or derivatives thereof of the present disclosure may also be used as a bioconjugate. Bioconjugates of the platelets or the platelet variants or derivatives thereof can be (i) a linker-based bioconjugate, where one or more of receptor protein or ligand or a molecule (e.g., on a cell surface) on the platelet or the platelet variant or derivatives thereof is linked to a cytotoxic agent via a linker (ii) the bioconjugate of the platelets or the platelet variants or derivatives thereof can be a linker free bioconjugate, where one or more of cytotoxic agents are directly conjugated to a receptor protein or ligand or a molecule on the cell surface of the platelets or the platelet variants or derivatives thereof without the aid of a linker, or (iii) the platelets or the platelet variants or derivatives thereof may imbibe the cytotoxic agents.

Bioconjugation techniques are well known to one of skill and the art and can be found, for example, in Bioconjugate Techniques, 3rd Edition (2013) by Greg T. Hermanson (ISBN 978-0-12-382239-0: Academic Press). “Bioconjugate Techniques” besides being a complete textbook and protocols-manual for biomolecular crosslinking, it is also an exhaustive and robust reference for conjugation strategies.

### Cytotoxic Agents-Drugs

The cytotoxic agents for the use in the embodiments of the present disclosure is a matter of choice for one of skill in the art depending on a disease which needs to be treated taking advantage of property of the platelet variants or derivatives to reach that target through the blood circulatory system. These cytotoxic agents may be selected from, but not limited to, an immunoinflammatory drug, a metabolic drug, neoplastic drug, a drug for curing autoimmune disease or any drug which the platelet variants or derivatives thereof of the present disclosure can deliver to a diseased target in need of that drug in human body. Once a symptom or a disease is identified appropriate drugs can be used as cytotoxic agents to cure that disorder. Drugs for delivery through the platelet variants or derivatives thereof can be selected from references such as Merck manual or by referring to Index to Drug-Specific Information on US Food & Drug Administration website: https://www.fda.gov/drugs/postmarket-drug-safety-information-patients-and-providers/index-drug-specific-information, or in a co-pending U.S. Patent Application Nos. 63/000,848 and Ser. No. 16/730,603, incorporated herein by reference in their entireties. In some embodiments, the drugs and/or compounds can be existing drugs or compounds, and in other embodiments, the drugs or compounds can be new or modified drugs and compounds.

### Disease or Disorders

Various diseases and disorders can be treated by the platelet variants or derivatives of the present disclosure because of their ability to circulate through the blood system and reaching a diseased target with ease as well as their ability to carry greater amount of drug payloads than conventional drug delivery means which uses the same methodology but without the platelet variants. These diseases or disorders are inclusive of but not limited to one or more of an immunoinflammatory disorder, a metabolic disorder, neoplastic disorder, autoimmune disorder or any disorder where the platelets or the platelet variants of the present disclosure can be delivered in human body. More specifically, the disorder could be one or more of rheumatoid arthritis, multiple sclerosis, type I diabetes mellitus, idiopathic inflammatory myopathy, systemic lupus erythematosus (SLE), myasthenia gravis, Grave's disease, dermatomyositis, polymyositis, Crohn's disease, ulcerative colitis, gastritis, Hashimoto's thyroiditis, asthma, psoriasis, psoriatic arthritis, dermatitis, systemic scleroderma and sclerosis, inflammatory bowel disease (IBD), respiratory distress syndrome, meningitis, encephalitis, uveitis, glomerulonephritis, eczema, atherosclerosis, leukocyte adhesion deficiency, Raynaud's syndrome, Sjorgen's syndrome, Reiter's disease, Beheet's disease, immune complex nephritis, IgA nephropathy, IgM polyneuropathies, immune-mediated thrombocytopenias e.g., ITP), acute idiopathic thrombocytopenic purpura, chronic idiopathic thembocytopenic purpura, hemolytic anemia, myasthenia gravis, lupus nephritis, atopic dermatitis, pemphigus vulgaris, opsoclonus-myoclonus syndrome, pure red cell aplasia, mixed cryoglobulinemia, ankylosing spondylitis, hepatitis C-associated cryoglobulinemic vasculitis, chronic focal encephalitis, bullous pemphigoid, hemophilia A, membranoproliferative glomerulonephritis, adult and juvenile dermatomyositis, adult polymyositis, chronic urticaria, primary biliary cirrhosis, neuromyelitis optica, Graves' dysthyroid disease, bullous pemphigoid, membranoproliferative glomerulonephritis, Churg-Strauss syndrome, juvenile onset diabetes, hemolytic anemia, atopic dermatitis, systemic sclerosis, Sjorgen's syndrome and glomerulonephritis, dermatomyositis, ANCA, aplastic anemia, autoimmune hemolytic anemia (AIHA), factor VIII deficiency, hemophilia A, autoimmune neutropenia, Castleman's syndrome, Goodpasture's syndrome, solid organ transplant rejection, graft versus host disease (GVHD), autoimmune hepatitis, lymphoid interstitial pneumonitis, HIV, bronchiolitis obliterans (non-transplant), Guillain-Barre Syndrome, large vessel vasculitis, giant cell (Takayasu's) arteritis, medium vessel vasculitis, Kawasaki's Disease, polyarteritis nodosa, Wegener's granulomatosis, microscopic polyangiitis (MPA), Omenn's syndrome, chronic renal failure, acute infectious mononucleosis, HIV and herpes virus associated diseases.

In some embodiments of the present disclosure, are methods for treating or ameliorating autoimmune diseases with the platelets or platelet variants or derivatives thereof (e.g., genetically engineered) that share at least one common receptor, ligand or an antigen with an endogenous autoantigenic cell population with, for example but not limited to, bone marrow derived platelets, nuclear and cell membrane phospholipid components, such as chromatin or ribonucleoprotein particles, insulin-producing cells, or autoantigenic cell population bearing autoantigenic peptides, such as but not limited to, GPIIb/IIIa, α-subunit of Acetylcholine Receptor (AChR), Aquaporin-4, ADAM metallopeptidase with thrombospondin type 1 motif 13 (ADAMTS13), Anti—N-methyl-D-aspartate receptor (anti-NMDAR), Phospholipase A2R that may be presented at different levels on different MHC molecules or otherwise and are targets for autoantibodies. Advantageously, the at least one common receptor, ligand or an antigen, whether endogenously or exogenously expressed on the platelets or platelet variants or derivatives thereof, specifically bind to autoantibodies that otherwise target the endogenous autoantigenic cell population. Binding of the autoantibodies to the platelets or platelet variants or derivatives thereof substantially frees the endogenous cell population (e.g., endogenous platelets) from damage caused by the autoantibodies. Upon elimination or decrease in the autoantibody population in a patient induced by the platelets or platelet variants or derivatives thereof (e.g., coupling of platelets or platelet variants with autoantibodies to form platelets or platelet variants-autoantibody complex), the endogenous cell population can perform their physiological role thereby eliminating or ameliorating autoimmune diseases. For example, platelets or platelet variants or derivative thereof can bind to autoantibodies against donor platelets thereby freeing the donor platelets to perform their physiological roles, as disclosed in co-pending U.S. application Ser. No. 17/213,796, filed on Mar. 26, 2021, which is expressly incorporated herein by reference it its entirety.

### Methods and Compositions

The present disclosure also provides a method and compositions of treating a disease or condition with a platelet variant population of the present disclosure. In some embodiments, the platelet variants can be administered to a patient in need thereof to augment or cure platelet-based deficiencies, as discussed in the forgoing. The method comprising administering to the patient in need there a therapeutic amount of platelet variants. In some embodiments, cytotoxic agents (e.g., a protein or a peptide, such as an antibody or a fragment thereof, a receptor or a portion thereof or a ligand or a fragment thereof, a drug or a prodrug) whether conjugated to a platelet variants via a linker (i.e., linker-based bioconjugates) or directly conjugated to the platelet variant or exogenously expressed in platelet variants or diffused into platelet variants (e.g., in granules) are administered in therapeutic amounts into a patient in need for treating a disease or condition with the cytotoxic agents. In some embodiments, the cytotoxic agents take advantage of the platelet variants systemic or rolling, adhesion, and aggregate formation capabilities to travel (roll) from a first location, where the platelet variants or derivatives thereof are administered, to a second location i.e., a diseased location, where the platelet variant or their derivatives adhere to and aggregate to mitigate or eliminate a disease. As an example, the present disclosure provides a method for treating a patient having a neoplasm comprising administering to said patient a therapeutically effective amount of an the non-naturally occurring platelet variant cell population or derivatives thereof or pharmaceutical composition described herein. The neoplasm is selected from, but not limited to, one or more of abdominal, bone, breast, digestive system, liver, pancreas, peritoneum, adrenal, parathyroid, pituitary, testicles, ovary, thymus, thyroid, eye, head and neck, central nervous system, peripheral nervous system, lymphatic system, pelvic, skin, soft tissue, spleen, thoracic region, and urogenital system. In some embodiments, the method comprises administering a second anti-cancer agent to the subject. In some embodiments, the second anti-cancer agent is a chemotherapeutic agent. The first and the second agent could be same or different depending upon the need of a patient. Thus the first and/or the second agent could be selected from one or more of an anti-CD20 therapeutic, an anti-IL-6 receptor therapeutic, an anti-IL-12/23p40 therapeutic, an immunosuppressant, an anti-Interferon beta-1a therapeutic, glatiramer acetate, an anti-alpha4-integrin therapeutic, fingolimod, an anti-BLyS therapeutic, CTLA-Fc, or an anti-TNF therapeutic.

For use in a method of treating a disease or condition with a cytotoxic agent (e.g., protein, peptide antibody or a drug), which inhibits RNA polymerase, the present disclosure also provides platelet variants or derivatives thereof in which the cytotoxic agent inhibits RNA polymerase and is prepared for administration with another therapeutic agent. The present disclosure also provides another therapeutic co-agent for use in a method of treating a disease or condition with a cytotoxic agent which inhibits RNA polymerase, wherein the other therapeutic co-agent is prepared for administration with the platelet variants or derivatives thereof.

In some embodiments, the CD34+ derived platelets or the platelet variants or derivatives thereof or lysates thereof are used to enrich platelet rich plasma (PRP) from a subject in need of the PRP treatment. Here, a combination of intact platelet variants or derivatives thereof or precursor cells making the platelet variants or derivatives thereof or lysates extracted therefrom can be used to enrich other source of platelet-enriched plasma, such as a donor-derived PRP. Optionally, it could include growth factors, cytokines or other agents from other sources, which complement the PRP-based therapeutic applications in a patient.

Typically, the platelets or the platelet variants or derivatives thereof are administered in a therapeutically effective dose. In view of the increased therapeutic efficacy, administration may occur less frequent as in treatment with conventional cell-based therapy or with bioconjugates and/or in a lower dose. Alternatively, in view of the increased tolerability, administration may occur more frequent as in treatment with conventional cell-based therapy or with bioconjugates and/or in a higher dose. Administration may be in a single dose or may e.g. occur every 3 to 4 hours, 1-4 times a day, 1-4 times a week, 1-4 times a month, possibly 1-7 times a week, or possibly administration occurs once every 3 or 4 weeks. As will be appreciated by the person skilled in the art, the dose of the platelets or the platelet variants or derivatives thereof, according to the present disclosure, may depend on many factors and optimal doses can be determined by the skilled person via routine experimentation. The bioconjugate is typically administered in a dose of 0.01-50 mg/kg body weight of the subject, 0.03-25 mg/kg or 0.05-10 mg/kg, or alternatively 0.1-25 mg/kg or 0.5-10 mg/kg. In some embodiments the first dosage is at a higher, same or at a lower concentration as the second dosage. The dosages can also be administered at different intervals of times.

The platelet variants or derivatives thereof, (e.g., platelet variants genetically programmed to produce a protein or polypeptide of interest, e.g., an Ipilimumab, secukinumab, trastuzumab antibody or a fragment thereof, or the platelet variant bioconjugates (e.g., platelet variants conjugated to a ligand or a receptor with or without a linker) may be used in vitro, ex vivo, or incorporated into pharmaceutical compositions and administered to individuals (e.g., human subjects) in vivo to treat, ameliorate, or prevent a disease or a disorder treatable by Ipilimumab, secukinumab, trastuzumab or with the platelet/platelet variant-bioconjugates of the present disclosure. A pharmaceutical composition will be formulated to be compatible with its intended route of administration (e.g., routes that are commonly followed during blood transfusion but performed with the platelets or the platelet variants or derivatives thereof of the present disclosure or through oral compositions generally include an inert diluent or an edible carrier). Other nonlimiting examples of routes of administration include parenteral (e.g., intravenous), intradermal, subcutaneous, oral (e.g., inhalation), transdermal (topical), transmucosal, and rectal administration. The pharmaceutical compositions compatible with each intended route are well known in the art.

Pharmaceutical composition comprising the platelets or the platelet variants or derivatives thereof of the present disclosure may be combined with a pharmaceutically acceptable carrier. Such a composition may contain, in addition to platelet variants or derivatives thereof, carriers, various diluents, fillers, salts, buffers, stabilizers, solubilizers, and other materials well known in the art. The characteristics of the carrier will depend on the route of administration. The pharmaceutical compositions for use in the disclosed methods may also contain additional therapeutic agents for treatment of the particular targeted disorder. For example, a pharmaceutical composition may also include other agents as disclosed herein. Such additional factors and/or agents may be included in the pharmaceutical composition to produce advantages of the therapeutic approaches disclosed herein, i.e., provide improved drug efficacy with reduced systemic toxicity.

In some embodiments, the administration of the composition comprising the platelets or the platelet variants or derivatives thereof, according to the present disclosure, is at a dose that is lower than the toxic dose (TD50) of the same bioconjugate but not comprising the platelet variants of the present disclosure. For example, the dose is at most 99-90%, 89-80%, 79-70%, 69-60%, 59-50%, 49-40%, 39-30%, 29-20%, 19-10%, 9-1% or 0.9-0.01% lower than the toxic dose (TD50) of the same bioconjugate but not comprising the platelets or the platelet variants or derivatives thereof of the present disclosure. Alternatively, the administration of the bioconjugate according to the present disclosure is at a dose that is higher than the TD50 of the same bioconjugate but not comprising platelet variants of the present disclosure. For example, the dose is at most 0.9 to 0.01%, 9-1%, 19-10%, 29-20%, 39-30%, 49-40%, 59-50%, 69-60%, 79-70%, 89-80%, 99-90% higher than the TD50 of the same bioconjugate but not comprising the platelets or the platelet variants or derivatives thereof of the present disclosure.

In some embodiments, the administration of the platelets or the platelet variants or derivatives thereof, according to the present disclosure, is at a dose that is lower than the ED50 of the same bioconjugate but not comprising the platelet variants of the present disclosure. For example, 99-90%, 89-80%, 79-70%, 69-60%, 59-50%, 49-40%, 39-30%, 29-20%, 19-10%, 9-1% lower than the effective dose (ED50) of the same bioconjugate but not comprising the platelets or the platelet variants or derivatives thereof of the present disclosure. Alternatively, the administration of the platelets or the platelet variants or derivatives thereof, according to the present disclosure, is at a dose that is higher than the ED50 of the same cell-based therapy or bioconjugate but not comprising the platelets or the platelet variants or derivatives thereof of the present disclosure. For example, the dose is at most 0.9 to 0.01%, 9-1%, 19-10%, 29-20%, 39-30%, 49-40%, 59-50%, 69-60%, 79-70%, 89-80%, 99-90% higher than the ED50 of the same bioconjugate but not comprising the platelets or the platelet variants or derivatives thereof of the present disclosure.

Herein, the term “therapeutic index” (TI) has the conventional meaning well known to a person skilled in the art, and refers to the ratio of the dose of drug that is toxic (i.e. causes adverse effects at an incidence or severity not compatible with the targeted indication) for 50% of the population (TD50) divided by the dose that leads to the desired pharmacological effect in 50% of the population (effective dose or ED50). Hence, TI=TD50/ED50. The therapeutic index may be determined by clinical trials or for example by plasma exposure tests. See also Muller, et al. Nature Reviews Drug Discovery 2012, 11, 751-761. At an early development stage, the clinical TI of a drug candidate is often not yet known. However, understanding the preliminary TI of a drug candidate is of utmost importance as early as possible since TI is an important indicator of the probability of the successful development of a drug. Recognizing drug candidates with potentially suboptimal TI at earliest possible stage helps to initiate mitigation or potentially re-deploy resources. At this early stage, TI is typically defined as the quantitative ratio between safety (maximum tolerated dose in mouse or rat) and efficacy (minimal effective dose in a mouse xenograft).

Herein, the term “therapeutic efficacy” denotes the capacity of a substance to achieve a certain therapeutic effect, e.g. reduction in tumor volume. Therapeutic effects can be measured determining the extent in which a substance can achieve the desired effect, typically in comparison with another substance under the same circumstances. A suitable measure for the therapeutic efficacy is the ED50 value, which may for example be determined during clinical trials or by plasma exposure tests. In case of preclinical therapeutic efficacy determination, the therapeutic effect of a bioconjugate (e.g. the platelets or the platelet variants or derivatives thereof), can be validated by patient-derived tumor xenografts in mice in which case the efficacy refers to the ability of the platelets or the platelet variants or derivatives thereof to provide a beneficial effect. Alternatively, the tolerability of the platelets or the platelet variants or derivatives thereof in a rodent safety study can also be a measure of the therapeutic effect.

Herein, the term “tolerability” refers to the maximum dose of a specific substance that does not cause adverse effects at an incidence or severity not compatible with the targeted indication. A suitable measure for the tolerability for a specific substance is the TD50 value, which may for example be determined during clinical trials or by other tests known to one of skill in the art.

The following examples are put forth so as to provide those of ordinary skill in the art with a complete disclosure and description of how to make and use the assay, screening, and therapeutic methods of the disclosure, and are not intended to limit the scope of what the inventors regard as their disclosure.

### EXAMPLES

Methods

### CD34+ Cell Expansion and Differentiation

On Day 0, a vial of 1×10⁶ or 5×10⁶ frozen peripheral blood CD34+ cells collected and purified by Fred Hutchinson Cooperative Center for Excellence in Hematology (CCEH) Hematopoietic Cell Processing Facility (Seattle, Wash.) were thawed and cultured in expansion medium (EXM) consisting of SFEM (STEMCELL Technologies) or StemMACS HSC Expansion Media and cocktail (Miltenyi Biotech), PEN/Strep (Thermo Fisher Scientific), glutamine (Thermo Fisher Scientific), lipids (Sigma), rhTPO, Flt-3, and SCF (PeproTech) at a concentration of 350,000 cells/mL. Cells suspension remained at 37° C., 5% CO₂, 20% O₂ for 2 days. On day 4, differentiation into MKs was induced by removing the EXM and replacing with differentiation medium (DM); SFEM, low density lipids (STEMCELL Technologies), SCF (PeproTech), rhTPO (STEMCELL Technologies), IL-9 (PeproTech), IL-6 (PeproTech), and SU6656 (SelleckChem). Cells were diluted to 350,000 cells/mL with DM and grown at 39° C., 7% CO₂, 20% O₂. On day 7 of differentiation, the cells were transferred back to 37° C., 5% CO₂, 20% O₂.

### Flow Cytometry Analysis

MKs and PLTs were counted using flow cytometry by staining for MK or PLT-specific surface markers, respectively. MKs were collected at various points during differentiation. The resulting cell suspensions were incubated with CD41 (APC, BioLegend #303710), CD34+ (FITC, BioLegend #343604), CD61 (FITC, BioLegend #336404), or CD42b (PE/Cy7, BioLegend #303916). Events were acquired on an Accuri C6 flow cytometer (BD Biosciences). PLTs were collected after a BioR run and stained with DRAQ5 nuclear stain and Calcein-AM (BioLegend #425201), as well as CD61 (VioBlue, Miltenyi #130-110-754), CD42a (PE, Miltenyi #130-100-966) and CD62p (PE-Vio770, Miltenyi #130-105-669). PLTs were designated as any event that was DRAQ5-negative, PLT-sized, CD61-positive, and CD42a-positive. CD62p-positive PLTs were considered activated. Samples were acquired on a MACSQuant Analyzer 10 (Miltenyi). The total number of PLTs or platelet variants in the bioreactor product was then calculated using the number of PLT events in the sample, volume run, and total volume of the bioreactor product. All data was analyzed with FlowJo Software, where cells were gated based on forward- and side-scatter parameters as determined by appropriate IgG controls.

### Ploidy

1×10⁶ cells/mL were collected, washed twice with cold PBS, and resuspended in 1 mL cold 70% EtOH and stored until processing at −20° C. Cells were resuspended in 10 μL ploidy blocking buffer (PBS containing 10% Fetal Bovine Serum [Thermo Fisher Scientific] and 2% Goat Serum [Thermo Fisher Scientific]). Cells were labeled with CD61 (APC, BioLegend #336412) or appropriate antibody control. Following incubation, cells were washed and resuspended in PBS containing 0.5% NP-40 (Sigma) and 100 μg/mL RNase A (Thermo Fisher Scientific), then labeled with propidium iodide (PI) (BioLegend). Flow cytometry analysis of samples was performed using an Accuri C6 Flow Cytometer (BD Biosciences) and data were analyzed with FlowJo Software. Gating was based on PI signal of CD61+ events, with area under the peak utilized to determine the percentage of DNA content.

### Confocal Imaging

MKs or resting platelets were fixed with 4% paraformaldehyde in PBS. Following fixation, cells were permeabilized and blocked with immunofluorescence blocking buffer (IFBB, 1% BSA, 10% goat serum in PBS). Cells were stained with the appropriate primary and, if necessary, secondary antibodies for the following signals F-actin (AlexaFluor 488 phalloidin [Thermo Fisher Scientific]), nuclear (DRAQ5 [Cell Signaling #4084L]), and β1-tubulin (AlexaFluor 488 secondary antibody [Thermo Fisher Scientific]). Alpha-granule staining was completed using specific antiserum for VWF (Novus Biologicals) and PF4 (Abcam) and counterstained with Hoechst (nuclear stain, Thermo Fisher Scientific) and CD42b-APC (surface stain, BioLegend #303912). For dense-granule labeling, specific antiserum for LAMP-1 (Abcam) and serotonin (Novus Biologicals) were used. Visualization of labeling was completed using appropriate secondary antibodies labeled with AlexaFluor 488 and AlexaFlour 555 (Thermo Fisher Scientific). Glass activated platelets were stained for F-actin. Resting platelets were stained for β1-tubulin and nuclei as described above. Resting platelets were also stained for CD61 (BioLegend #336402) and visualized using AlexaFluor 555 secondary antibody. Following, the proper staining coverslips were mounted using Aqua-Poly/Mount (Thermo Fisher Scientific). Visualization of samples was completed using a Zeiss Meta 880 laser scanning confocal micrograph equipped with 40× Pln Apo 40×/1.3 oil DICII and 63× Pln Apo 63×/1.4 oil DICII objective. Micrographs were analyzed using Fiji ImageJ.

### Megakaryocyte Lysate Analysis

Mature MKs, defined as viable, nucleated, and positive for CD41 and CD61 were collected on Day 8 and lysed using a freeze/thaw method. Briefly, pelleted MKs were stored at −80° C. overnight, then thawed the following day for 5 min at 37° C. After the final thaw, 10 μl DNAse I (Qiagen) was added for 30 min then centrifuged at 3000×g for 10 min. The resulting supernatant centrifuged again at 5000×g for 20 min and stored at −20° C. Samples of the CD34+-derived MK lysate and PLTMax Human Platelet Lysate (Sigma #SCM141) were diluted in PBS (1:20) and analyzed using Luminex technology (Eve Technologies Corporation, Calgary, AB, Canada). Cytokine concentration in each sample was calculated from its fluorescent intensity, based on a standard curve. A cytokine was designated as “present” if the concentration was above zero and not extrapolated, and “not present” if these conditions were not satisfied.

### Electron Microscopy

Cells were placed into 37° C. fixative (2.5% formaldehyde glutaraldehyde in 0.1M sodium cacodylate buffer, pH 7.4) and pelleted at 200×g and incubated at RT for 1 hour. Supernatant was removed and replaced with 1 mL of 0.1M sodium cacodylate. After 5 minutes, the sodium cacodylate was removed and the sample was stored at 4° C. until further processing. Sample processing and imaging was completed using the Harvard Medical School Electron Microscopy Core. Briefly, MKs were fixed with 1.5% glutaraldehyde in 0.1 M cacodylate buffer, pH 7.4, for 8 hours. Ultrathin sections were stained with uranyl acetate and lead citrate and examined with a Tecnai G2 Spirit BioTWIN transmission electron microscope (Hillsboro, Oreg.) at an accelerating voltage of 80 kV, and images were recorded with an AMT 2k CCD camera and accompanying proprietary in-house software (Danvers, Mass.).

### Platelet Bioreactor

The custom-built bioreactor for making platelet variants consists of two channels with 5 μm pores in the top polycarbonate membrane (Sterlitech). The tubing, syringes, and reactor are primed with 70% ethanol in water to sterilize the setup, then rinsed with 70 mL of degassed PBS, followed by degassed StemSpan ACF Medium (STEMCELL Technologies) with 1% PEN/STREP. For the cell seeding, a syringe containing 3.5 mL of cell solution is connected to the MK inlet and perfused at 0.5 mL/min for 6 minutes. The perfusion volume is replaced with 3 mL of fresh media. Media with 1% PEN/STREP (16 mL) is then recirculated at 800 s⁻¹ for 3 hours using two NE-4000 Programmable 2 channel syringe pumps (New Era Pump Systems). At hours 2 and 3, samples are taken and analyzed by FACS to determine the total number of platelet variants in the circulating volume. At 3 hours, samples are fixed 1:3 in 4% paraformaldehyde for further analysis.

### Bioreactor Platelet Variant Concentration

The platelet variants produced by the bioreactor was concentrated in bioreactor media supplemented with a 1:10000 dilution of 1 mg/mL PGE1 and centrifuged at 1250×g for 30 minutes with brake set to 3. Cells were resuspended with PAS and concentration was calculated according to readings taken from Macsquant Analyzer 10 (Miltenyi).

### Drug Loading

The therapeutic monoclonal antibody, ipilimumab, was purchased from Selleck Chemical and labeled with Cy5.5 (Lumiprobe) and filtered with 40K Zeba desalting column (Thermo Fisher Scientific) per vendors instructions. Antibody concentration was determined by measurement with Pierce 660 protein quantification (Thermo Fisher Scientific). Prior to loading with the Cy5.5-labeled ipilimumab, the PLTs/PLCs were characterized as previously mentioned. For each loading experiment, varying concentrations of PLTs/PLCs and Cy5.5-labeled ipilimumab reaction was allowed to catalyze for 1 hour at 37° C. in dark. After loading, PGE1 (final concentration 100 ng/mL) was added. Cells were centrifuged twice for 10 minutes at 800×g brake 3, then resuspended and characterized with the PLT/PLC markers.

### Immunohistochemistry

Organs were fixed in 10% formalin and stored in 70% ethanol until processing. Organs were sent out to be paraffin embedded, sliced, and mounted on slides. Slides were deparaffinized in plastic coplin jars (Thomas #62101) in 2 changes of histology grade xylene (Sigma-Aldrich #534056-500) for 5 minutes each. They were then hydrated in 2 changes of 100% ethanol for 3 minutes each, 95% ethanol for 1 minute, then 70% ethanol for one minute. They were rinsed in H₂O for 45 seconds then immersed in IHC-Tek epitope retrieval solution (IHC World #IW-1001) and steamed for 40 minutes with the cap off. Slides were cooled for 20 minutes then rinsed in immunofluorescence blocking buffer (IFBB, 0.5 g BSA, 5 mL goat serum, 45 mL PBS) 2 times for 5 minutes each. Slides were stained as described in confocal imaging.

### Biodistribution Studies

All murine experiments were conducted with the assistance of Charles River Labs (CRL). Washed PLTs/PLCs were isolated as previously described. Approximately 1.5×10⁸ wash PLTs/PLCs were labeled with ˜5 uM Xenolight DiR (PerkinElmer) and incubated at 37° C. for 10 minutes. The wash and bioreactor PLTs/PLCs were concentrated and resuspended at 5×10⁷ PLTs/PLCs/mL. Control suspensions of dye-free PLTs/PLCs were isolated in a similar fashion, stopping and resuspending before the dye step. Biodistribution studies were conducted on NCG mice (lacking Prkdc and Il2rg), with the assistance from CRL. Experimental conditions were as follows: PAS buffer control, un-dyed washed PLCs, un-dyed CD34+ PLTs, Xenolight dyed washed PLTs/PLCs, and Xenolight CD34+ bioreactor PLCs. 5 mice per condition were examined and each mouse received a 200 uL injection via tail vein. Mice were imaged with an IVIS system over a 60-minute time frame with 1 image taken every 15 minutes. At the end of the 60-minute examination, mice were sacrificed, and tissue was extracted for endpoint imagining analysis with IVIS. Image analysis was conducted by Living Image Software (Perkin Elmer).

### Results

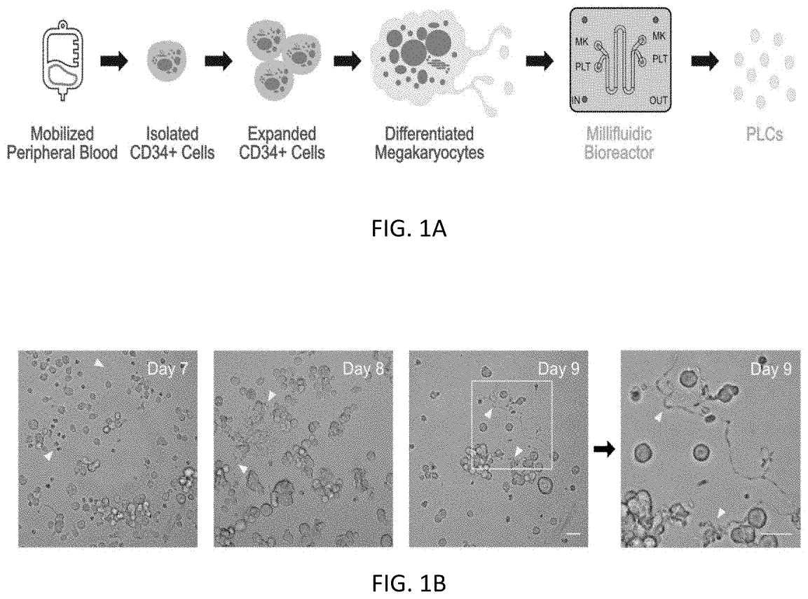

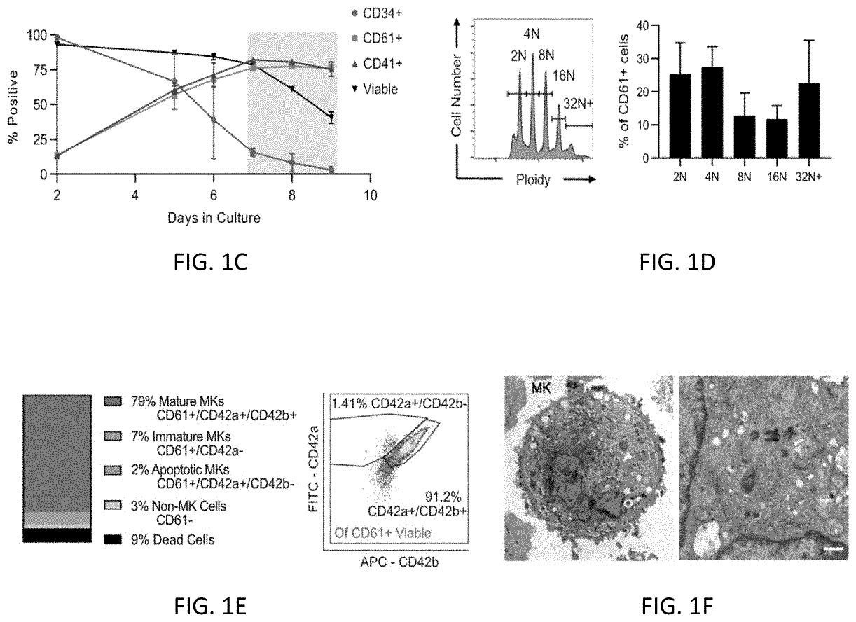

Advantageously, it was discovered a novel method to differentiate CD34+ progenitor cells (e.g., peripheral blood-derived CD34+ cells), which rapidly differentiates the CD34+ progenitor cells into mature, primary-like MKs. Advantageously, the primary-like MKs can be scaled in a bioreactor to produce platelet variants or derivatives thereof. This rapid method generates about or greater than 36.4±10.3 CD61+ MKs per starting CD34+ progenitor cell; this finding is believed to represents the highest MK yield yet reported using primary HSCs. The MKs produced by the rapid method exhibit high purity with the CD61+CD42a+CD42b+ population representing 79% of total cells (FIG. 1E). Unexpectedly, as provided by the rapid methodologies of the present disclosure, the MKs formed proPLTs as early as day 5 and released PLTs decreasing the culture time and reagent cost associated with longer differentiation protocols. FIG. 1F shows transmission electron microscope micrographs of MKs at increasing magnifications (890×, and 9300×, respectively). Arrows indicate standard MK characteristics (multilobed nuclei, microtubule rings, alpha-granules, invaginated membrane system, and Golgi stacks). Scale bars are 6 μm (890×), and 600 nm (9300×), respectively.

These conclusions are based on in-depth characterization using a set of “gold standard” metrics, confirming the primary-like quality of MKs and platelets or platelet variants or derivatives thereof generated by the methods disclosed herein. Robust characterization of the MKs and platelets or platelet variants verified that MKs differentiated using the methodologies disclosed herein mimic the behavior of primary MKs, making them ideal candidates for producing MK and platelets or platelet variants or derivatives thereof for therapeutic use or for clinical applications.

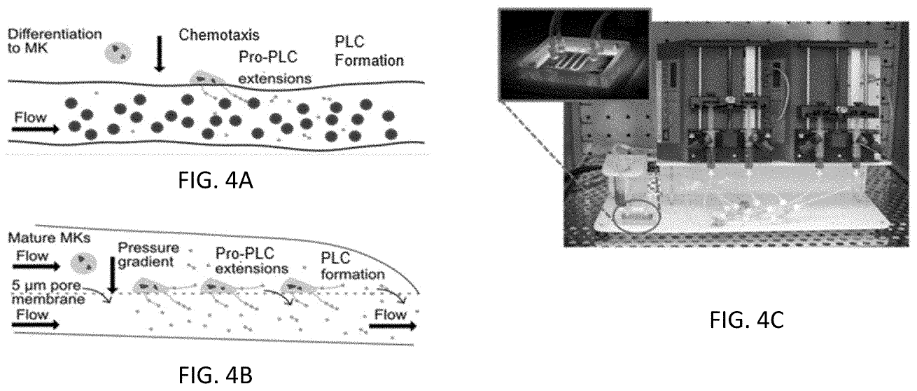

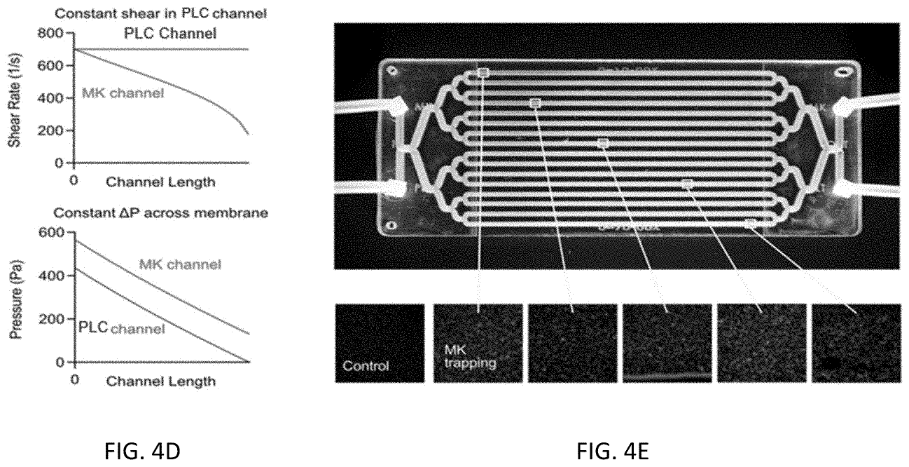

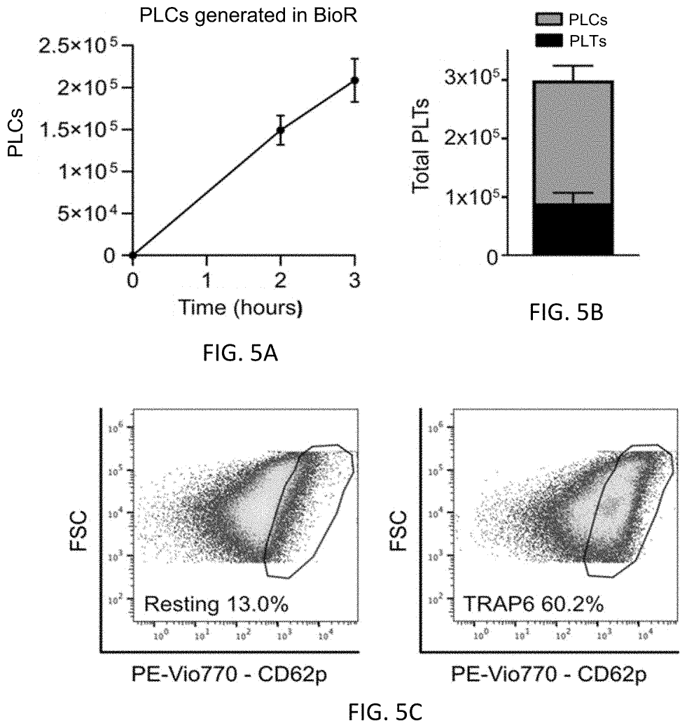

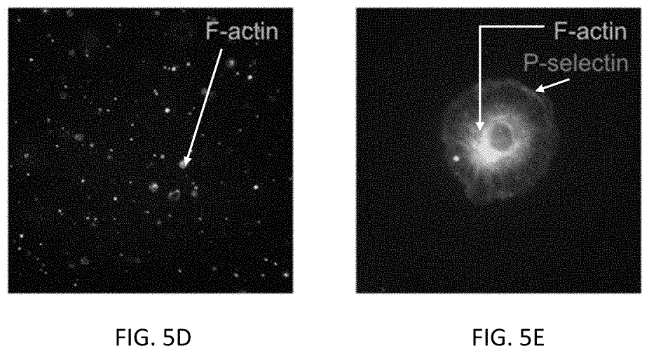

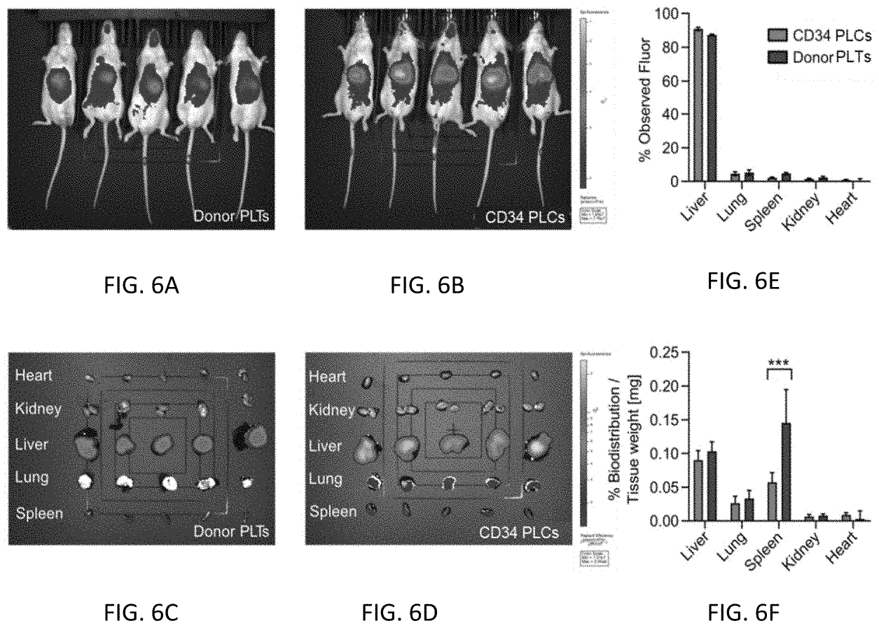

In some embodiments, the CD34+ progenitor cell-derived MKs are engineered in a bioreactor to produce platelet variants or derivatives thereof, schematically illustrated in FIGS. 4A-4D. The bioreactor comprises two channels. CD34+ progenitor cell-derived MKs are seeded in the top channel and retained, allowing proplatelets and PLTs/PLCs to pass through for collection. After demonstrating that a single bioreactor produces functional platelet variants or derivatives thereof (e.g., see FIGS. 5A-5E), a scaled version was developed that significantly increases the production of platelet variants (PLCs) or derivatives thereof over time as seen in the biodistribution experiments (FIGS. 6A-6F). This bioreactor also has the potential to be multiplexed, further scaling up platelet variants (PLCs) production.

The differentiation protocol described herein is suitable for generating MKs from any source of CD34+ cells from peripheral blood. Unlike bone marrow or UCB, peripheral blood represents a minimally invasive source of CD34+ cells with the potential for obtaining multiple vials from the same donor, thereby reducing variability in cell source. Peripheral blood-derived culture methods are also less expensive than iPSC methods, improving the ability of investigators to generate MKs when cost is a consideration.

It was observed that, by passive uptake, antibody payload co-localizes with PF4-expressing alpha granules and, thus, has the potential to be selectively secreted upon platelet activation. Ipilimumab, an anti-CTLA4 antibody and FDA approved drug for the treatment of metastatic melanoma, was used in this study to highlight the potential for platelet: drug cargo to be selectively delivered to primary and secondary tumors as a result of the phenomenon of tumor cell-induced platelet aggregation (TCIPA). Antibody-based therapeutics are highly efficacious but often produce immune-related adverse events (irAEs) that lead to disorders such as Irritable Bowel Syndrome and Crohn's Disease, co-morbidities that are common and often-times predictive of positive clinical outcomes. Selective delivery of this class of drugs in platelets could lessen these symptoms dramatically by reducing the Effective Dose (ED) required to ameliorate disease. Packing therapeutics into platelets may increase the pharmacokinetic window for systemically administered drugs as well as bias the in vivo biodistribution of drugs to be localized at sites of disease pathology.

The current study shows identical biodistribution patterns between CD34+ progenitor cells derived platelets or platelet variants and washed platelets from whole blood in immunocompromised mice (FIGS. 6A-6F). It has been shown previously that, despite the reduction in B, T, NK-cells, and other cells necessary for mounting a full response to xenobiotics, infused platelets will be detected at primary sites of clearance (namely, the liver and spleen) as soon as 1 hour post infusion (Kaplan and Saba, 1978). The CD34+-derived platelets used in this study followed the same biodistribution pattern as peripheral blood-derived platelets presumably due to preferential clearance in the liver by Kupffer cells and hepatocytes.

Stem cell-derived, ex vivo generated platelets or platelet variants are a viable, stable, and, under the right conditions, sterile alternative with great potential for scale-up and commercial feasibility. Drug-loaded versions of these platelets or platelet variants have the potential to be an ideal cellular therapeutic for a broad swatch of diseases as evidenced by enhanced pharmacokinetics, biodistribution, and efficacy. Therefore, the only feasible path to reach the clinic requires stem cell-derived cellular products which substantially resemble in morphology, biomarker expression, and function to that of endogenous platelets, as disclosed herein.

### CD34+ Progenitor Cell Differentiation Protocol Generates Mature MKs

Using previously described methods as a starting point, the present disclosure provides a robust differentiation protocol to generate mature MKs from CD34+ progenitor cells. This two-stage protocol includes an expansion/maintenance stage (Stage (0); day−2 to day 0) and a differentiation phase (Stage I; day 0 to day 12+), which encompasses the time during which MKs have matured and produce proPLTs and PLTs (day 6 to day 10). FIG. 1A is an overall schematic of the process demonstrating the instant protocol (PBG protocol) in addition to generation of platelets utilizing the bioreactor.

MKs produced by this optimized protocol were characterized after 9 days in differentiation media. Cells exhibited features consistent with mature MKs as observed by electron microscopy (EM), including multilobed nuclei, alpha-granules, and dense granules (FIG. 1B). These cells were also larger than CD34+ HSCs (13.6±4.2 μm diameter relative to <7 μm¹⁶) consistent with differentiation into MKs. CD34+ progenitor cell cultures also produced proplatelets (proPLTs) as early as day 5 of the differentiation protocol (data not shown). These results encouraged further characterization of differentiated cells to verify that they phenocopy primary MKs.

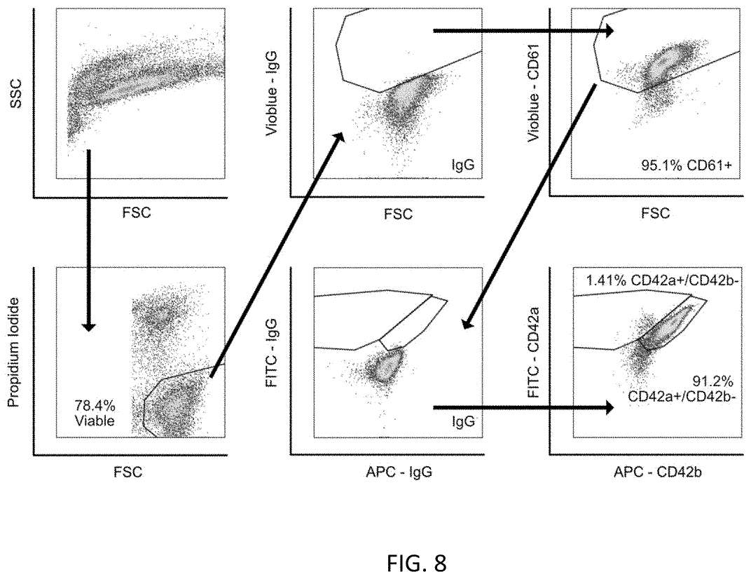

In-depth analysis of differentiated MKs was performed using a compiled set of “gold standard” MK characterization metrics (Table 1). As expected during differentiation, HSCs subjected to the instant protocol exhibited decreasing CD34+ expression, with cultures reaching a minimum of CD34+ cells on day 9 (FIG. 1C). These cultures also exhibited viable cells exceeding 75% of MK-sized events through day 7 (FIG. 1C), which decreased post day-7, coinciding with the start of PLT release. This observation is consistent with the observation that apoptosis may play a role in PLT formation. The instant CD34+ progenitor cells also exhibited substantial commitment to the MK lineage by day 9, where 79% of cells were CD61+CD42a+CD42b+ (representing mature MKs) and an additional 7% were CD61+CD42a−CD42b− (representing MK progenitors) (FIG. 1E). FIG. 8 demonstrates the gating strategy for these cell populations.

Differentiated cells also exhibited size and ploidy characteristics of mature MKs. The majority of CD61+ cells were 4N, with a 22.6±12.9% having DNA content of 32N or higher (FIG. 1D). Interestingly, the size and ploidy profiles of the MKs derived as presently described resemble those of MKs differentiated from UCB, which are smaller and have lower ploidy than MKs differentiated from peripheral blood.

TABLE 1
MK characterization prepared by the process of the present disclosure.

| | Days to | | Cytoskeletal | Granule | Surface | |
|---|---|---|---|---|---|---|
| Source | Maturation | Morphology | Organization | Content | Analysis | Other |
| PB CD34+ | Day 7-9 | Bright field IF | B1-tubulin | PF4 | CD34+ | Ploidy |
| | | Microscopy | F-actin | VWF | CD41 | Luminex |
| | | Megakaryocyte | | Serotonin | CD42a | Platelet Yield |
| | | EM | | LAMP1 | CD42b | Agonist Activation |
| | | | | VEGF* | CD61 | Contact Activation |
| | | | | bFGF* | CD62p | Thrombin Generation |
| | | | | PDGF* | GPVI | Assay |
| | | | | FGF* | PAC1 | Microfluidic Assay |
| | | | | EGF* | Annexin V | Platelet |
| | | | | | | Biodistribution |

IF = Immunofluorescent
*Analysis by Luminex

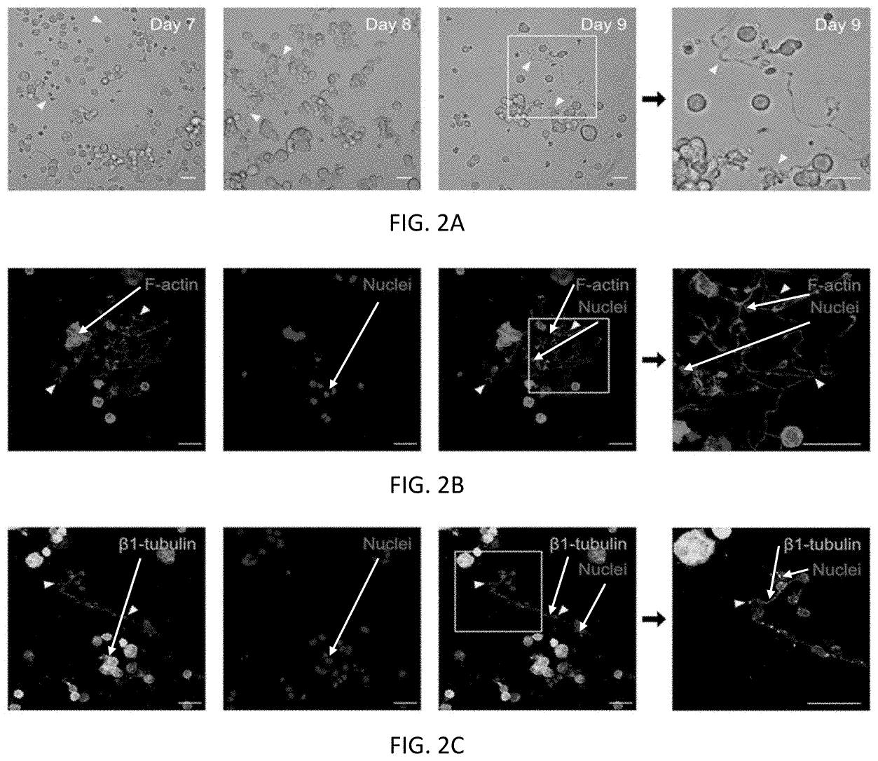

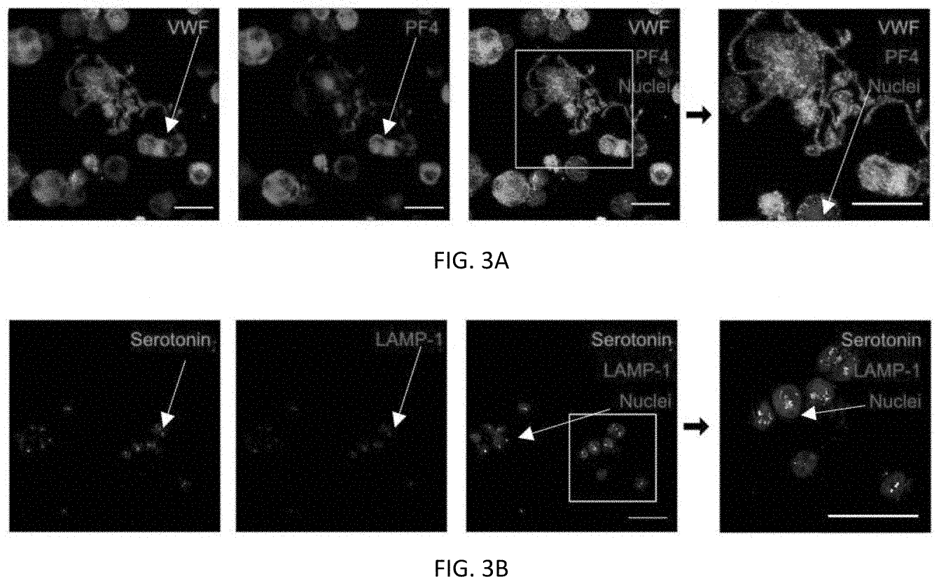

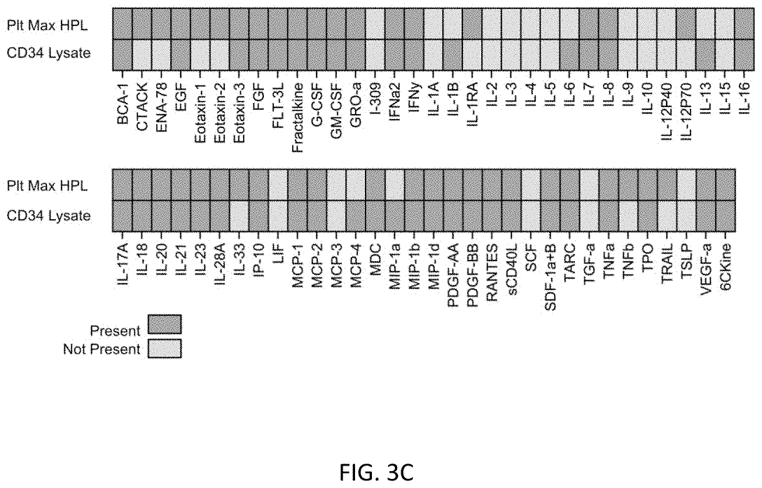

It was next determined that CD34+ progenitor cells derived MKs exhibited markers of normal PLT generation. In addition to the characteristic ultrastructural features described above (FIG. 1B), MKs also produced proPLTs consistently over time in ulture as observed through light microscopy (FIG. 2A: proPLTs indicated by arrows). Normal proPLT formation requires the presence of an F-actin networks (involved in proPLT spreading and bifurcation) and β1-tubulin (a MK- and PLT-specific tubulin isoform involved in proPLT extension and PLT structure and function). As expected, MKs derived as described herein exhibited intense F-actin and β1-tubulin staining, both in cell bodies and in extending proPLTs (FIGS. 2B and 2C; proPLTs indicated by arrows). ProPLTs were also anucleate, as indicated by absence of a DRAQ5 signal (FIGS. 2B and 2C). The ability to generate proplatelets led to investigation of the presence of α-granules in the cells. The development of platelet α-granules began in the MKs, where the cells derived from the CD34+ progenitor cells stained positively for PF4 and VWF, two factors that are uniquely secreted by platelets and cells important in maintaining and disrupting hemostasis (FIG. 3A). MKs also expressed dense granule markers lysosome-associated membrane protein-1 (LAMP-1) and serotonin (FIG. 3B).

It was also determined that differentiated MKs expressed multiple analytes essential to normal PLT function. Primary PLT alpha-granules are enriched for signaling proteins including epidermal growth factor (EGF), vascular endothelial growth factor (VEGF) and PLT-derived growth factor (PDGF). PLTs and megakaryocytes also absorb circulating thrombopoietin (TPO). The presence of these proteins in CD34+ progenitor cells derived MK lysate was assessed using a Luminex assay, with human platelet lysate (HPL) as a positive control. As expected, CD34+ progenitor cells derived MK lysate contained EGF, PDGF-AA, PDGF-BB, and VEGF-α (FIG. 3C). Lysate from the instant MKs for expression of cytokines and other relevant chemokines and growth factors (FIG. 3C) was profiled, which showed substantial overlap in protein profiles between the lysate from the instant MKs and HPL.

MKs Derived from CD34+ Progenitor Cells Produce Novel Platelet Variants in a Bioreactor

The high quality of the instant MKs derived from CD34+ progenitor cells as described herein led to ask whether they were suitable for producing PLTs in the setting of a novel millifluidic bioreactor. This platelet bioreactor mimics the myeloid vascular niche, to which maturing MKs migrate via chemotaxis from the proliferative osteoblastic niche. Once in the vascular niche, MKs extend proPLTs through capillary walls and into circulation, where prePLTs and PLTs are released (FIG. 4A). The bioreactor recapitulates this microenvironment using two 12.5-cm microfluidic channels—one for seeding MKs and one for collecting platelets—separated by a polycarbonate membrane (FIGS. 4B and 4C). This membrane contains 5-μm pores that retain MKs while allowing proPLTs and generated platelet variants to extend into the bottom channel. To initially examine whether CD34+-derived MKs were able to produce platelet variants, this bioreactor was seeded with 230,000±2,500 MKs and monitored platelet variant production via flow cytometry over the course of three hours. FIG. 4D illustrates a graph showing that the sheer rate in the PLT channel stays constant throughout the channel length and a graph showing that the change in pressure between the PLT and MK channels stays constant across their length. FIG. 4E shows the 16-channel version of the bioreactor and fluorescent images showing even seeding of MKs throughout its channels and their lengths.

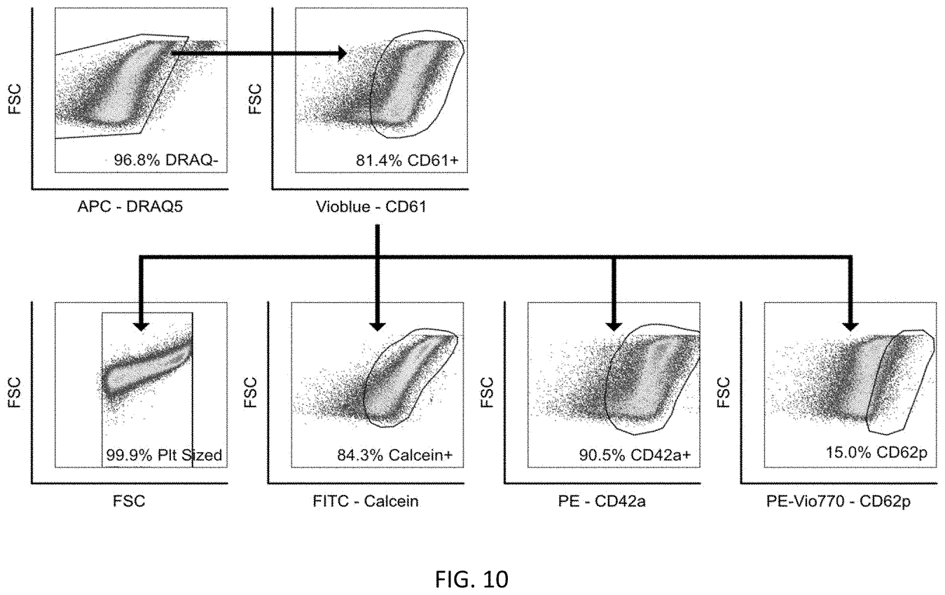

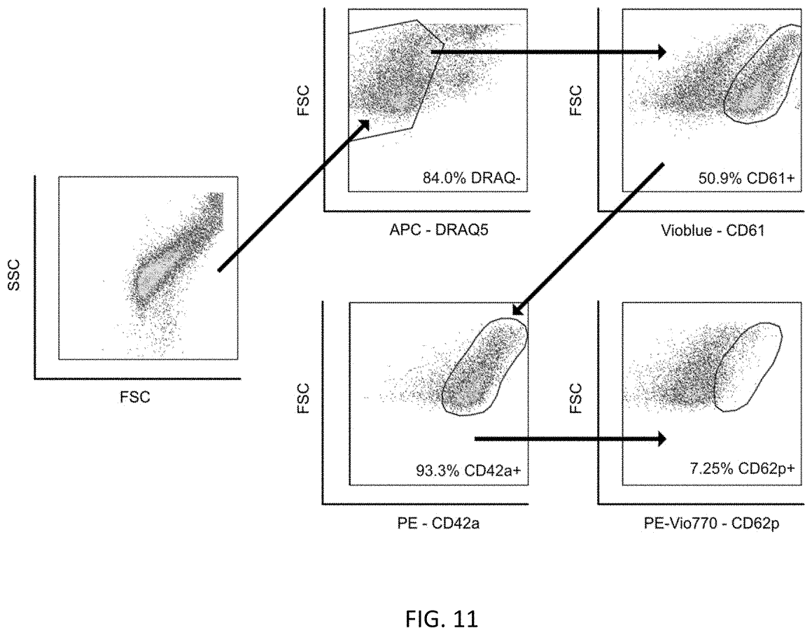

During this time, production of platelet variants proceeded at a near-constant rate (FIG. 5A) with 208,667±25,775 CD41+/CD61+/DRAQ5− platelet variants being generated beyond the 89,733±17,570 PLTs already present in the MK-seeding media (representing platelet variants generated in static culture) (FIG. 5B). This number represents a yield of 0.9 PLTs per MK seeded during the three-hour bioreactor run and 0.4 PLTs per MK produced in static culture, for an overall yield of 1.3 platelet variants per MK. Platelet variants were counted using a gating strategy where platelet-sized anucleated events expressed CD61, CD42a, and calcein-AM (FIG. 10). Additionally, the resting status of the platelets were examined by CD62p surface expression, 13% of platelets immediately removed from the bioreactor expressed CD62p on the surface.

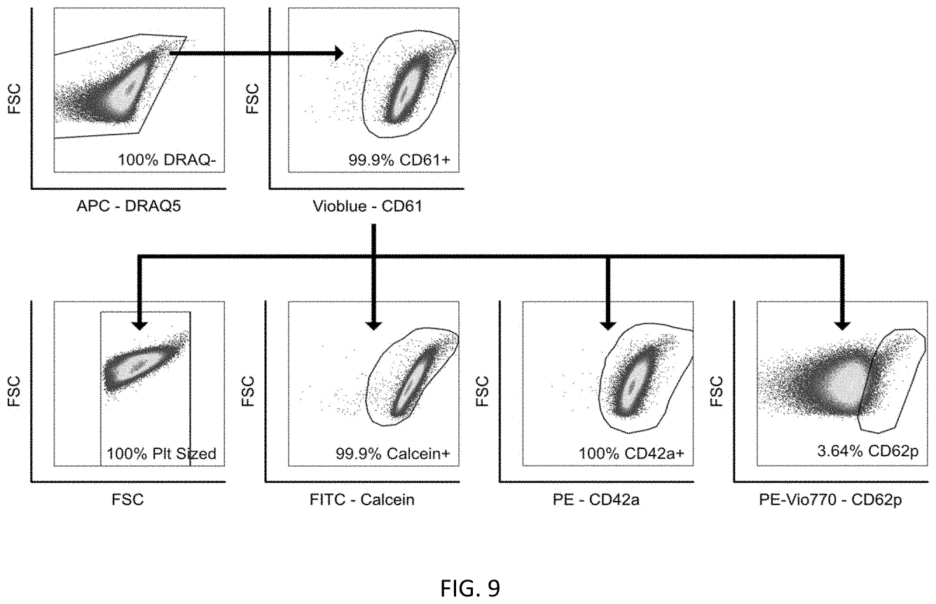

To further analyze the bioreactor-derived platelet variants, their ability to activate under a variety of conditions was assessed. When examined under activation via a chemical agonist, TRAP6, bioreactor-derived platelet variants exhibited an increased extracellular exposure of CD62p as compared to mock-treated PLTs (FIG. 5C), indicating the bioreactor-derived platelet variants are capable of rapidly mobilizing α-granules to the platelet surface to encourage cell to cell interactions. In FIG. 5D, the cytoskeleton and morphology were examined by immunofluorescence. β1-tubulin staining was completed to visually demonstrate the morphology and cytoskeleton of our bioreactor-derived platelets. Further visual assessment was completed by assessing the ability of the platelet variants to exhibit formation of actin-containing lamellipodia and filopodia (FIG. 5E). These results indicate that the platelet variants produced in the bioreactor by CD34+ progenitor cell-derived MKs exhibit low basal levels of activation but are capable of robust activation upon stimulation with an agonist. FIG. 8 shows characterization of mature MKs. FIG. 9 shows characterization of donor platelets. FIG. 10 shows characterization of platelet variants.

### Biodistribution of the Bioreactor Produced Platelet Variants

In order to first look at how bioreactor produced platelet variants might behave in a similar manner to donor platelets, the biodistribution of each was compared in an immune-compromised mouse model, NCG mice produced by Charles River Labs. These mice are like the NOD-SCID models of mice, ablating the Prkdc and Il2rg from mice in order to eliminate maturation of B-cells, T-cells, and Natural Killer cells and act as a permissive host for human cell engraftment.

In order to compare the biodistribution of bioreactor generated platelet variants to donor PLTs in vivo, each was loaded with the lipophilic fluorescent dye Xenolight DiR. mice was then injected with either dye alone, dye-loaded donor PLTs, or dye-loaded bioreactor platelet variants via tail vein. Mice were imaged every 15 minutes using IVIS imaging to assess systemic dye distribution. After 1 hour, IVIS imaging indicated most of the PLT product traveled to the liver and intestinal cavity, independent of whether sourced from CD34+ cells or from healthy donors (FIG. 6A). Murine heart, kidney, liver, lungs, and spleen were harvested and assessed for dye biodistribution by IVIS fluorescence imaging at the end (FIG. 6B). Organs from bioreactor-platelet variants or donor-PLT—treated mice exhibited approximately equal levels of fluorescence in the same tissue (FIGS. 6C and 6D). The overall distribution as measured by Mann-Whitney and Wilcoxon statistics indicated no statistical difference between the two conditions. The percent biodistribution was normalized to tissue weights and calculated a significant increase in splenic contribution in the donor-PLT treated mice as compared to the CD34+ progenitor cells derived platelets (by Sidak's multiple comparisons test) (FIG. 6E). This difference could be attributed to the fact that the CD34+-derived sample was 95% pure whereas washed donor platelets had 100% CD61 expression. The resulting impurities, while minute, may lead to a greater percentage of the sample becoming cleared in the liver, thus leading to the relatively greater splenic clearance seen in the donor platelet condition (data not shown). The vast majority of cells, whether donor PLTs or CD34+-derived platelets or platelet variants homed to the liver at upwards of 90% of input product, while the heart had one of the lowest signals, indicating that circulating blood was largely devoid of Xenolight DiR signal, possibly implying that platelets or platelet variants were embedded in the smaller vessels of the lung or in the process of being cleared by the liver and spleen. Regardless, there were few significant differences between donor and CD34+-derived platelets or platelet variants, highlighting that biodistribution of both cells is largely equivalent. The percent of biodistribution and tissue weight was compared and resulted in significant differences in the spleen, as shown in the bar graph in FIG. 6F.

### Platelets or Platelet Variants as a Potential Drug Delivery Mechanism

The increase in platelet or platelet variant production has allowed for the exploration of potential applications of the bioreactor-derived platelets or platelet variants, including the capacity to bind biologic and small molecule drugs for potential application in drug delivery. It was hypothesized that another activity of CD34+-derived platelets or platelet variants could be utilized, namely endocytosis of surrounding macromolecules in the circulation. In this way, in vitro platelets or platelet variants could package and deliver an antibody drug to a specific, distal location in the body, such as a tumor. As a proof-of-concept, the study focused on the monoclonal antibody ipilimumab, which targets cytotoxic T-lymphocyte—associated antigen 4 (CTLA-4) and used as an immune checkpoint inhibitor in melanoma. The capabilities of bioreactor PLTs in drug uptake and retention were determined.

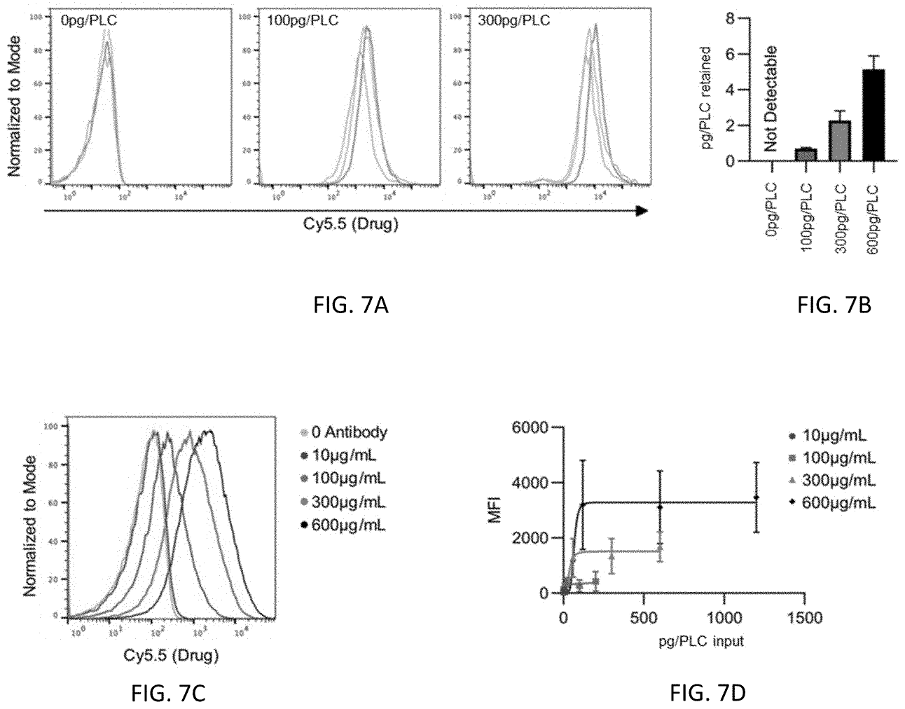

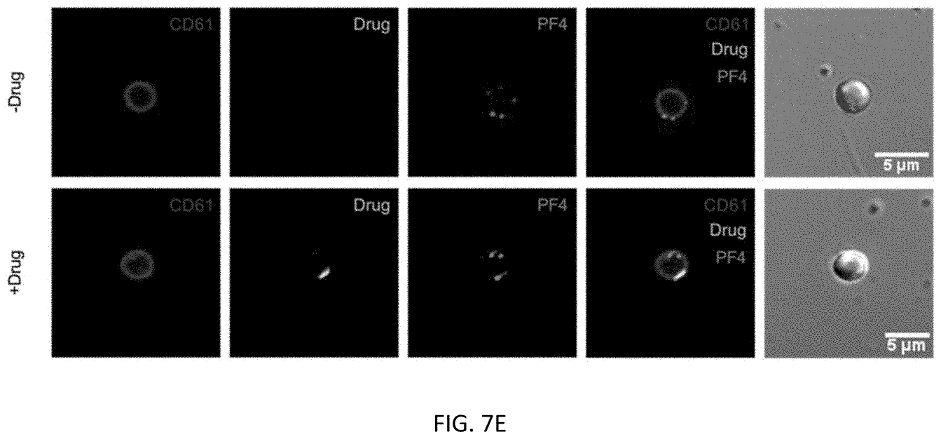

Platelets or bioreactor-generated platelet variants were incubated with ipilimumab conjugated to Cy5.5, and drug loading was assessed by flow cytometry (FIG. 7A). Ipilimumab retention was observed at varying input concentrations, with overlaid histogram plots illustrating the retention of the drug after multiple wash steps. The loaded platelets or platelet variants were lysed and analyzed for Cy5.5 fluorescence by plate reader, ultimately leading to a calculated payload of roughly 1 to 6 pg/PLT or variant thereof, increasing in a dose-dependent manner with respect to input antibody concentration (FIG. 7B). These data indicate that a drug (e.g., a monoclonal antibody) in solution can be taken up by CD34+-derived platelets or platelet variants, remain associated with the cell through multiple wash steps, and detected by a variety of techniques.

Next, the loading limitations of platelets or platelet variants was examined to define the saturating input dosage required for maximal drug loading. The loading dynamics of a static concentration of platelets or platelet variants was examined when exposed to increasing concentrations of Ipilimumab. CD34+ platelets or platelet variants exhibited the ability to retain antibody across a range of 100 ug/mL to 600 ug/mL, with increasing efficiency of loading as concentration of drug was increased (FIG. 7C). Drug and platelets or platelet variants concentrations were manipulated to examine how the input ratio of drug: platelets or platelet variants would affect possible saturation points of drug. Increasing platelets or platelet variants concentration from a low of 10⁴ PLTs or variants thereof to a high of 10⁶ PLTs or variants thereof were exposed to varying drug concentrations to fully elucidate the loading capabilities (FIG. 7D). An inflection point was observed at less than 100 pg/PLT or variants thereof input concentration in order to achieve maximal loading (EC₉₀=70.042 pg/PLT or variants thereof for 300 ug/ml antibody concentration; EC₉₀=94.993 pg/PLT for 600 ug/ml antibody concentration) (FIG. 7D).

Having determined that platelets or platelet variants are able to associate and retain a given antibody-drug, it was sought to visualize its subcellular localization. There is colocalization between drug and CD61, indicating drug is likely adhering to membranous surface areas, as well as drug and Platelet Factor 4 (PF4), indicating likely uptake into the alpha-granules (FIG. 7E). The fact that drug deposition appears to be both intracellular and extracellular indicates a unique loading-and-retention mechanism, which opens a host of therapeutic possibilities to bioreactor-derived platelets or platelet variants, as they could readily engulf a therapeutic and be delivered as a short-lived or long-lived or stable vehicles by which cargo may be administered to distal locations in the body.

All patents and publications mentioned in this specification are herein incorporated by reference to the same extent as if each independent patent and publication was specifically and individually indicated to be incorporated by reference.

1-47. (canceled)

48. A method for generating megakaryocytes from progenitor stem cells comprising at least two stages:
(i) performing a stage zero (0) comprising an expansion and maintenance stage of the progenitor stem cells; and,
(ii) performing a stage one (I) comprising a differentiation phase wherein the differentiation phase comprises differentiating the progenitor stem cells in step (i) for a period sufficient to generate matured megakaryocytes, wherein the matured megakaryocytes are positive for one or more of CD61, CD42a, and CD42b.

49. The method of claim 48, wherein the progenitor stem cells are CD34+ progenitor stem cells.

50. The method of claim 48, further comprising culturing the matured megakaryocytes in a bioreactor subject to one or more of shear stress, mechanical strain and pulsed electromagnetic field.

51. The method of claim 48, further comprising differentiating the matured megakaryocytes to pro-platelets or platelets.

52. A composition comprising platelets produced by the method of claim 48, wherein the platelets are used in treating a disease or a disorder in subject.

53. The composition of claim 52, wherein the disease or disorder is selected from one or more of an immunoinflammatory disorder, a metabolic disorder, a neoplastic disorder, an autoimmune disorder, viral or bacterial-induced disorder.

54. The composition of claim 52, further comprising a cytotoxic agent selected from one or more of an antibody, a nucleic acid, a protein or a polypeptide, or a drug or a prodrug and a combination thereof.

55. A method for generating genetically engineered megakaryocytes comprising at least:
(i) genetically engineering progenitor stem cells for forming genetically engineered megakaryocytes;
(ii) performing a stage zero (0) comprising an expansion and maintenance stage of the genetically engineered progenitor stem cells, and
(iii) performing a stage one (I) comprising a differentiation phase wherein the differentiation phase comprises differentiating the genetically engineered progenitor stem cells in step (i) for a period sufficient to generate matured engineered megakaryocytes, wherein the matured engineered megakaryocytes are positive for one or more of CD61, CD42a, and CD42b.

56. The method of claim 55, wherein the progenitor stem cells are CD34+ progenitor stem cells.

57. The method of claim 56, wherein the genetically engineered progenitor stem cells express one or more exogenous nucleic acids encoding for one or more of a therapeutic protein(s) or a polypeptide(s), a receptor, or a fragment thereof, selected from one or more of a cell-surface receptor or transmembrane receptor, an ion channel-linked receptor, a G-protein-coupled receptor, an enzyme-linked receptor or an internal receptor and a combination thereof.

58. The method of claim 57, wherein the therapeutic protein(s) or the polypeptide(s) is selected from one or more of an antibody or a fragment thereof, a growth factor, a hormone, an antigen, a cytokine and a combination thereof.

59. The method of claim 55, further comprising one or more of differentiating the genetically engineered megakaryocytes to engineered pro-platelets or platelets and culturing the genetically engineered megakaryocytes in a bioreactor.

60. A method for generating platelet variants from progenitor stem cells comprising at least:
(i) performing an expansion and maintenance stage of the progenitor stem cells comprising culturing the progenitor stem cells;
(ii) performing a differentiation stage wherein the differentiation stage comprises differentiating the progenitor stem cells in step (i) for a period sufficient to generate matured megakaryocytes, wherein the matured megakaryocytes are positive for one or more of CD61, CD42a, and CD42b; and
(iii) passaging said matured megakaryocytes through a bioreactor wherein the matured megakaryocytes generate platelet variants.

61. The method of claim 60, wherein the progenitor stem cells are CD34+ progenitor stem cells.

62. The method of claim 61, wherein the CD34+ progenitor stem cells comprise an exogenous nucleic acid encoding for a protein.

63. The method of claim 62, wherein the protein is expressed in the platelet variants.

64. A method for generating platelet variants from progenitor stem cells for administration into a subject in need thereof comprising passaging megakaryocytes through a bioreactor, wherein the megakaryocytes are positive for one or more of CD61, CD42a, and CD42b.

65. The method of claim 64, wherein the progenitor stem cells are CD34+ progenitor stem cells.

66. The method of claim 65, wherein a bioreactor gradient in the bioreactor generates platelet variants and wherein the platelet variants do not exhibit uncontrolled growth or tumor formation in vivo.

67. A method for generating genetically engineered platelet variants from progenitor stem cells for administration into a subject in need thereof comprising:
(i) genetically engineering the progenitor stem cells and differentiating to produce genetically engineered megakaryocytes, wherein the genetically engineered megakaryocytes are positive for one or more of CD61, CD42a, and CD42b; and,
(ii) passaging the genetically engineered megakaryocytes through a bioreactor subject to one or more of shear stress, mechanical strain and pulsed electromagnetic field.
